(19) United States
(12) Patent Application Publication
(10) Pub. No.: US 2011/0046210 A1
(43) Pub. Date: Feb. 24, 2011

(54) SUBSTITUTED THIOPENECARBOXAMIDES AS IKK-BETA SERINE-, THREONINE-PROTEIN KINASE INHIBITORS

(75) Inventors: David Festus Charles Moffat, Oxfordshire (GB); Stephen John Davies, Oxfordshire (GB)

Correspondence Address: BANNER & WITCOFF, LTD., 1100 13th STREET, N.W., SUITE 1200, WASHINGTON, DC, 20005-4051, US

(73) Assignee: CHROMA THERAPEUTICS LTD., Abingdon, Oxfordshire (GB)

(21) Appl. No.: 12/989,271

(22) PCT Filed: Apr. 23, 2009
(86) PCT No.: PCT/GB09/01051

§ 371 Date: Oct. 22, 2010

(30) Foreign Application Priority Data
Apr. 26, 2008 (GB) 0807642.4
Aug. 27, 2008 (GB) 0815550.9

(51) Int. Cl. A61K 31/381; C07D 333/38; A61P 37/00; A61P 29/00; A61P 35/00; A61P 19/02; A61P 17/06; A61P 1/00; A61P 1/04; A61P 3/10; A61P 25/28; C12N 9/99

(52) U.S. Cl. 514/447; 549/69; 435/184

(57) ABSTRACT

Compounds of formula (IA) or (IB) are IKK inhibitors useful in the treatment of autoimmune and inflammatory diseases: wherein R₇ is hydrogen or optionally substituted (C₁-C₆)alkyl; A is an optionally substituted aryl or heteroaryl of 5-13 ring atoms; Z is a radical of formula R₁C(R₂)(R₃)NH—Y-L¹-X1-(CH₂)_{z}— wherein R₁ is a carboxylic acid group (—COOH), or an ester group which is hydrolysable by one or more intracellular esterase enzymes to a carboxylic acid group; and R₂ and R₃ independently represent the side chain of a natural or non-natural alpha amino acid but neither of R₂ and R₃ is hydrogen, or R₂ and R₃ taken together with the carbon atom to which they are attached form a C₃-C₇ cycloalkyl ring, and z, Y, L¹ and X¹ are as defined in the claims.

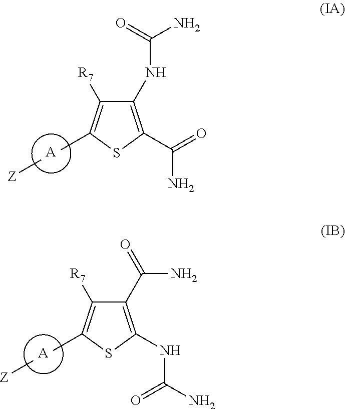

This invention relates to thiophene carboxamides characterised by the presence in the molecule of an α,α-disubstituted glycine ester motif, to compositions containing them, to processes for their preparation and to their use in medicine as IKK inhibitors for the treatment of autoimmune and inflammatory diseases, including chronic obstructive pulmonary disease, asthma, rheumatoid arthritis, psoriasis, inflammatory bowel disease, Crohn's disease, ulcerative colitis, multiple sclerosis, diabetes, atopic dermatitis, graft versus host disease, systemic lupus erythematosus. The compounds are also of use in the treatment of proliferative disease states, such as cancer.

### BACKGROUND OF THE INVENTION

The expression of many pro-inflammatory genes is regulated by the transcriptional activator nuclear factor-kB (NF-kB). These transcription factors have been suspected since their discovery to play a pivotal role in chronic and acute inflammatory diseases. It now seems that aberrant regulation of NF-kB could also underlie autoimmune diseases and different types of cancer.

Examples of genes dependent on the activation of NF-kB include: the cytokines tumour necrosis factor TNF-α, interleukin (IL)-6, IL-8 and IL-1β; the adhesion molecules E-selectin, intercellular adhesion molecule (ICAM)-1 and vascular cell adhesion molecule (VCAM)-1; and the enzymes nitric oxide synthase (NOS) and cyclooxygenase (COX)-2. NF-kB normally resides in the cytoplasm of unstimulated cells as an inactive complex with a member of the IkB inhibitory protein family. However, upon cellular activation, IkB is phosphorylated by the IkB kinase (IKK) and is subsequently degraded. Free NF-kB then translocates to the nucleus where it mediates pro-inflammatory gene expression.

There are three classical IkB's: IkBα, IkBβ and IkBε; all of which require the phosphorylation of two key serine residues before they can be degraded. Two major enzymes IKK-α and IKK-β appear to be responsible for IkB phosphorylation. Dominant-negative (DN) versions of either of these enzymes (where ATP binding is disabled by the mutation of a key kinase domain residue) were found to suppress the activation of NE-kB by TNF-α, IL-1β and LPS. Importantly IKK-β DN was found to be a far more potent inhibitor than IKK-α DN (Zandi, E Cell, 1997, 91, 243). Furthermore, the generation of IKK-α and IKK-β deficient mice established the requirement of IKK-β for activation of NF-kB by proinflammatory stimuli and reinforced the dominant role of IKK-β suggested by biochemical data. Indeed it was demonstrated that IKK-α was dispensable for NF-kB activation by these stimuli (Tanaka, M.; Immunity 1999, 10, 421). Thus, inhibition of IKK-β represents a potentially attractive target for modulation of immune function and hence the development of drugs for the treatment of auto-immune diseases.

### BRIEF DESCRIPTION OF THE INVENTION

This invention makes available a class of thiophene carboxamides which are potent and selective inhibitors of IKK isoforms, particularly IKK-β. The compounds are thus of use in medicine, for example in the treatment of a variety of proliferative disease states, such as conditions related to the hyperactivity of IKK, as well as diseases modulated by the NF-kB cascade. In addition, the compounds of the invention are useful for the treatment of stroke, osteoporosis, rheumatoid arthritis and other inflammatory disorders. The compounds are characterised by the presence in the molecule of an α,α-disubstituted glycine ester motif which is hydrolysable by an intracellular carboxylesterase. Compounds of the invention having the lipophilic α,α-disubstituted glycine ester motif cross the cell membrane, and are hydrolysed to the acid by the intracellular carboxylesterases. The polar hydrolysis product accumulates in the cell since it does not readily cross the cell membrane. Hence the IKK inhibitory activity of the compound is prolonged and enhanced within the cell. The compounds of the invention are related to the IKK inhibitors encompassed by the disclosure in International Patent Application No. WO 2004063186 but differ therefrom in that the present compounds have the α,α-disubstituted glycine ester motif referred to above.

The compounds of the invention are also related to those disclosed in our co-pending International Patent Application No. PCT/GB2007/004114. The latter compounds have an α-monosubstituted glycine ester motif which also enables the compounds to cross the cell membrane into the cell where they are hydrolysed to the corresponding acid by intracellular carboxylesterases. However, that publication does not suggest that α,α-disubstituted glycine ester conjugates can be hydrolysed by intracellular carboxylesterases. In fact, it appears that the ability of the intracellular carboxyl esterases, principally hCE-1, hCE-2 and hCE-3, to hydrolyse α,α-disubstituted glycine esters has not previously been investigated.

The general concept of conjugating an α-mono substituted glycine ester motif to a modulator of an intracellular enzyme or receptor, to obtain the benefits of intracellular accumulation of the carboxylic acid hydrolysis product is disclosed in our International Patent Application WO 2006/117567. However, this publication does not suggest that α,α-disubstituted glycine ester conjugates can be hydrolysed by intracellular carboxylesterases. As mentioned above, it appears that the ability of the intracellular carboxyl esterases, principally hCE-1, hCE-2 and hCE-3, to hydrolyse α,α-disubstituted glycine esters has not previously been investigated.

### DETAILED DESCRIPTION OF THE INVENTION

According to the present invention there is provided compound of formula (IA) or (IB), or a salt thereof:

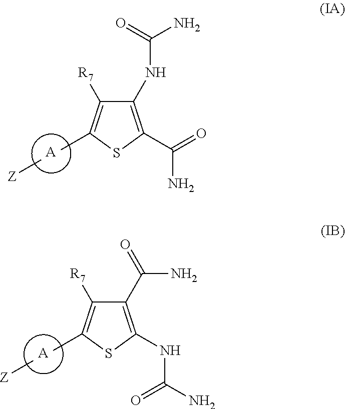

wherein
R₇ is hydrogen or optionally substituted (C₁-C₆)alkyl;
A is optionally substituted aryl or heteroaryl ring or ring system of 5-13 atoms;
Z is a radical of formula R₁C(R₂)(R₃)NH—Y-L¹-X¹—(CH₂)_{z}— wherein:
z is 0 or 1;
Y is a bond, —C(═O)—, —S(═O)₂—, —C(═O)NR₇—, —C(═S)—NR₇, —C(═NH)NR₇ or —S(═O)₂NR₇— wherein R₇ is hydrogen or optionally substituted C₁-C₆ alkyl;
L¹ is a divalent radical of formula -(Alk¹)ₘ(Q)ₙ(Alk²)ₚ- wherein
m, n and p are independently 0 or 1,
Q is (i) an optionally substituted divalent mono- or bicyclic carbocyclic or heterocyclic radical having 5-13 ring members, or (ii), in the case where both m and p are 0, a divalent radical of formula —X²-Q¹- or -Q¹-X²— wherein X² is —O—, S— or NR^{A}— wherein R^{A} is hydrogen or optionally substituted C₁-C₃ alkyl, and Q¹ is an optionally substituted divalent mono- or bicyclic carbocyclic or heterocyclic radical having 5-13 ring members,
Alk¹ and Alk² independently represent optionally substituted divalent C₃-C₇ cycloalkyl radicals, or optionally substituted straight or branched, C₁-C₆ alkylene, C₂-C₆ alkenylene, or C₂-C₆ alkynylene radicals which may optionally contain or terminate in an ether (—O—), thioether (—S—) or amino (—NR^{A}—) link wherein R^{A} is hydrogen or optionally substituted C₁-C₃ alkyl; and
   X¹ represents a bond; —C(═O); or —S(═O)₂—; —NR₄C(═O)—, —C(═O)NR₄—, —NR₄C(═O)NR₅—, —NR₄S(═O)₂—, or —S(═O)₂NR₄— wherein R₄ and R₅ are independently hydrogen or optionally substituted C₁-C₆ alkyl.
   R₁ is a carboxylic acid group (—COOH), or an ester group which is hydrolysable by one or more intracellular esterase enzymes to a carboxylic acid group; and
   R₂ and R₃ independently represent the side chain of a natural or non-natural alpha amino acid but neither of R₂ and R₃ is hydrogen, or R₂ and R₃ taken together with the carbon atom to which they are attached form a C₃-C₇ cycloalkyl ring.

Compounds of formula (IA) or (IB) above may be prepared in the form of salts, especially pharmaceutically acceptable salts, N-oxides, hydrates, and solvates thereof. Any claim to a compound herein, or reference herein to “compounds of the invention”, “compounds with which the invention is concerned”, “compounds of formula (IA) or (IB)” and the like, includes salts, N-oxides, hydrates, and solvates of such compounds.

In another broad aspect, the invention provides the use of a compound of formula (IA) or (IB) as defined above, or an N-oxide, salt, hydrate or solvate thereof in the preparation of a composition for inhibiting the activity of IKK, especially IKK-β, as well as diseases modulated by the NF-kB cascade.

The compounds with which the invention is concerned may be used for the inhibition of IKK, especially IKK-β, activity in vitro or in vivo.

Pharmaceutical compositions comprising a compound of the invention together with one or more pharmaceutically acceptable carriers and excipients, also form part of the invention.

In one aspect of the invention, the compounds of the invention may be used in the preparation of a composition for the treatment of neoplastic/proliferative, autoimmune or inflammatory disease, particularly those mentioned above in which IKK, especially IKK-β, activity plays a role.

In another aspect, the invention provides a method for the treatment of the foregoing disease types, which comprises administering to a subject suffering such disease an effective amount of a compound of formula (IA) or (IB) as defined above.

### Terminology

As used herein, the term “(Cₐ-C_{b})alkyl” wherein a and b are integers refers to a straight or branched chain alkyl radical having from a to b carbon atoms. Thus when a is 1 and b is 6, for example, the term includes methyl, ethyl, n-propyl, isopropyl, n-butyl, isobutyl, sec-butyl, t-butyl, n-pentyl and n-hexyl.

As used herein the term “divalent (Cₐ-C_{b})alkylene radical” wherein a and b are integers refers to a saturated hydrocarbon chain having from a to b carbon atoms and two unsatisfied valences.

As used herein the term “(Cₐ-C_{b})alkenyl” wherein a and b are integers refers to a straight or branched chain alkenyl moiety having from a to b carbon atoms having at least one double bond of either E or Z stereochemistry where applicable. The term includes, for example, vinyl, allyl, 1- and 2-butenyl and 2-methyl-2-propenyl.

As used herein the term “divalent (Cₐ-C_{b})alkenylene radical” means a hydrocarbon chain having from a to b carbon atoms, at least one double bond, and two unsatisfied valences.

As used herein the term “(Cₐ-C_{b})alkynyl” wherein a and b are integers refers to straight chain or branched chain hydrocarbon groups having from a to b carbon atoms and having in addition one triple bond. This term would include for example, ethynyl, 1-propynyl, 1- and 2-butynyl, 2-methyl-2-propynyl, 2-pentynyl, 3-pentynyl, 4-pentynyl, 2-hexynyl, 3-hexynyl, 4-hexynyl and 5-hexynyl.

As used herein the term “divalent (Cₐ-C_{b})alkynylene radical” wherein a and b are integers refers to a divalent hydrocarbon chain having from a to b carbon atoms, and at least one triple bond.

As used herein the term “carbocyclic” refers to a mono-, bi- or tricyclic radical having up to 16 ring atoms, all of which are carbon, and includes aryl and cycloalkyl.

As used herein the term “cycloalkyl” refers to a monocyclic saturated carbocyclic radical having from 3-8 carbon atoms and includes, for example, cyclopropyl, cyclobutyl, cyclopentyl, cyclohexyl, cycloheptyl and cyclooctyl.

As used herein the unqualified term “aryl” refers to a mono-, bi- or tri-cyclic carbocyclic aromatic radical, and includes radicals having two monocyclic carbocyclic aromatic rings which are directly linked by a covalent bond. Illustrative of such radicals are phenyl, biphenyl and napthyl.

As used herein the unqualified term “heteroaryl” refers to a mono-, bi- or tri-cyclic aromatic radical containing one or more heteroatoms selected from S, N and O, and includes radicals having two such monocyclic rings, or one such monocyclic ring and one monocyclic aryl ring, which are directly linked by a covalent bond. Illustrative of such radicals are thienyl, benzthienyl, furyl, benzfuryl, pyrrolyl, imidazolyl, benzimidazolyl, thiazolyl, benzthiazolyl, isothiazolyl, benzisothiazolyl, pyrazolyl, oxazolyl, benzoxazolyl, isoxazolyl, benzisoxazolyl, isothiazolyl, triazolyl, benztriazolyl, thiadiazolyl, oxadiazolyl, pyridinyl, pyridazinyl, pyrimidinyl, pyrazinyl, triazinyl, indolyl and indazolyl.

As used herein the unqualified term “heterocyclyl” or “heterocyclic” includes “heteroaryl” as defined above, and in its non-aromatic meaning relates to a mono-, bi- or tri-cyclic non-aromatic radical containing one or more heteroatoms selected from S, N and O, and to groups consisting of a monocyclic non-aromatic radical containing one or more such heteroatoms which is covalently linked to another such radical or to a monocyclic carbocyclic radical. Illustrative of such radicals are pyrrolyl, furanyl, thienyl, piperidinyl, imidazolyl, oxazolyl, isoxazolyl, thiazolyl, thiadiazolyl, pyrazolyl, pyridinyl, pyrrolidinyl, pyrimidinyl, morpholinyl, piperazinyl, indolyl, morpholinyl, benzfuranyl, pyranyl, isoxazolyl, benzimidazolyl, methylenedioxyphenyl, ethylenedioxyphenyl, maleimido and succinimido groups.

A “divalent phenylene, pyridinylene, pyrimidinylene, or pyrazinylene radical” is a benzene, pyridine, pyrimidine or pyrazine ring, with two unsatisfied valencies, and includes 1,3-phenylene, 1,4-phenylene, and the following:

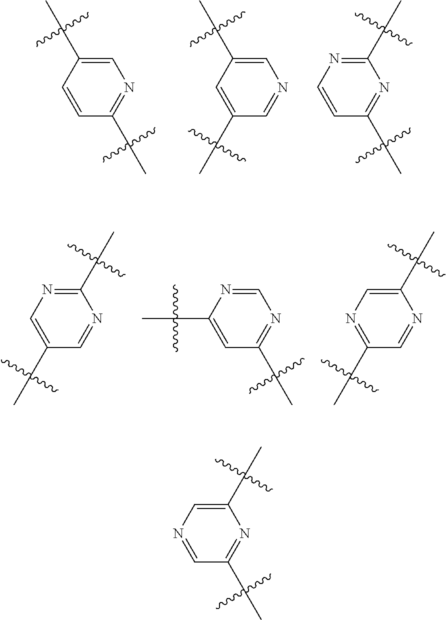

Unless otherwise specified in the context in which it occurs, the term “substituted” as applied to any moiety herein means substituted with up to four compatible substituents, each of which independently may be, for example, (C₁-C₆)alkyl, (C₁-C₆)alkoxy, hydroxy, hydroxy(C₁-C₆)alkyl, mercapto, mercapto(C₁-C₆)alkyl, (C₁-C₆)alkylthio, phenyl, halo (including fluoro, bromo and chloro), trifluoromethyl, trifluoromethoxy, nitro, nitrile (—CN), oxo, —COOH, —COOR^{A}, —COR^{A}, —SO₂R^{A}, —CONH₂, —SO₂NH₂, —CONHR^{A}, —SO₂NHR^{A}, —CONR^{A}R^{B}, —SO₂NR^{A}R^{B}, —NH₂, —NHR^{A}, —NR^{A}R^{B}, —OCONH₂, —OCONHR^{A}, —OCONR^{A}R^{B}, —NHCOR^{A}, —NHCOOR^{A}, —NR^{B}COOR^{A}, —NHSO₂OR^{A}, —NR^{B}SO₂OH, —NR^{B}SO₂OR^{A}, —NHCONH₂, —NR^{A}CONH₂, —NHCONHR^{B}, —NR^{A}CONHR^{B}, —NHCONR^{A}R^{B}, or —NR^{A}CONR^{A}R^{B} wherein R^{A} and R^{B} are independently a (C₁-C₆)alkyl, (C₃-C₆) cycloalkyl, phenyl or monocyclic heteroaryl having 5 or 6 ring atoms, or R^{A} and R^{B} when attached to the same nitrogen atom form a cyclic amino group (for example morpholino, piperidinyl, piperazinyl, or tetrahydropyrrolyl). An “optional substituent” may be one of the foregoing substituent groups.

The term “side chain of a natural or non-natural alpha-amino acid” refers to the group R^{Y} in a natural or non-natural amino acid of formula NH₂—CH(R^{Y})—COOH.

Examples of side chains of natural alpha amino acids include those of alanine, arginine, asparagine, aspartic acid, cysteine, cystine, glutamic acid, histidine, 5-hydroxylysine, 4-hydroxyproline, isoleucine, leucine, lysine, methionine, phenylalanine, proline, serine, threonine, tryptophan, tyrosine, valine, α-aminoadipic acid, α-amino-n-butyric acid, 3,4-dihydroxyphenylalanine, homoserine, α-methylserine, ornithine, pipecolic acid, and thyroxine.

Natural alpha-amino acids which contain functional substituents, for example amino, carboxyl, hydroxy, mercapto, guanidyl, imidazolyl, or indolyl groups in their characteristic side chains include arginine, lysine, glutamic acid, aspartic acid, tryptophan, histidine, serine, threonine, tyrosine, and cysteine. When R₂ or R₃ in the compounds of the invention is one of those side chains, the functional substituent may optionally be protected.

The term “protected” when used in relation to a functional substituent in a side chain of a natural alpha-amino acid means a derivative of such a substituent which is substantially non-functional. For example, carboxyl groups may be esterified (for example as a C₁-C₆ alkyl ester), amino groups may be converted to amides (for example as a NHCOC₁-C₆ alkyl amide) or carbamates (for example as an NHC(═O)OC₁-C₆ alkyl or NHC(═O)OCH₂Ph carbamate), hydroxyl groups may be converted to ethers (for example an OC₁-C₆ alkyl or a O(C₁-C₆ alkyl)phenyl ether) or esters (for example a OC(═O)C₁-C₆ alkyl ester) and thiol groups may be converted to thioethers (for example a tert-butyl or benzyl thioether) or thioesters (for example a SC(═O)C₁-C₆ alkyl thioester).

Examples of side chains of non-natural alpha amino acids include those referred to below in the discussion of suitable R₂ and R₃ groups for use in compounds of the present invention.

As used herein the term “salt” includes base addition, acid addition and quaternary salts. Compounds of the invention which are acidic can form salts, including pharmaceutically acceptable salts, with bases such as alkali metal hydroxides, e.g. sodium and potassium hydroxides; alkaline earth metal hydroxides e.g. calcium, barium and magnesium hydroxides; with organic bases e.g. N-methyl-D-glucamine, choline tris(hydroxymethyl)amino-methane, L-arginine, L-lysine, N-ethyl piperidine, dibenzylamine and the like. Those compounds (IA) or (IB) which are basic can form salts, including pharmaceutically acceptable salts with inorganic acids, e.g. with hydrohalic acids such as hydrochloric or hydrobromic acids, sulphuric acid, nitric acid or phosphoric acid and the like, and with organic acids e.g. with acetic, tartaric, succinic, fumaric, maleic, malic, salicylic, citric, methanesulphonic, p-toluenesulphonic, benzoic, benzenesulfonic, glutamic, lactic, and mandelic acids and the like. For a review on suitable salts, see Handbook of Pharmaceutical Salts: Properties, Selection, and Use by Stahl and Wermuth (Wiley-VCH, Weinheim, Germany, 2002).

It is expected that compounds of the invention may be recovered in hydrate or solvate form, or in the case of some structures in N-oxidised form, and such forms are expected to have the activity of the non-hydrated, non-solvated or non-N-oxidised forms. The term ‘solvate’ is used herein to describe a molecular complex comprising the compound of the invention and a stoichiometric amount of one or more pharmaceutically acceptable solvent molecules, for example, ethanol. The term ‘hydrate’ is employed when said solvent is water.

Compounds of the invention which contain one or more actual or potential chiral centres, because of the presence of asymmetric carbon atoms, can exist as enantiomers or as a number of diastereoisomers with R or S stereochemistry at each chiral centre. The invention includes all such enantiomers and diastereoisomers and mixtures thereof.

As mentioned, the esters of the invention are converted by intracellular esterases to the carboxylic acids. Both the esters and carboxylic acids may have IKK-β kinase inhibitory activity in their own right. The compounds of the invention therefore include not only the ester, but also the corresponding carboxylic acid hydrolysis products.

In the compounds of the invention, the variable substituents and groups will now be discussed in more detail:

### The Substituent R₇

R₇ is hydrogen or optionally substituted (C₁-C₆)alkyl, such as methyl, ethyl or n- or iso-propyl. Currently preferred is when R₇ is hydrogen.

### The Ring or Ring System A

Ring A is optionally substituted divalent aryl or heteroaryl of 5-13 atoms, for example a divalent phenylene, pyridinylene, pyrimidinylene, or pyrazinylene radical. Currently preferred is 1,3-phenylene.

The Radical —Y-L¹-X¹—[CH₂]_{z}—

This radical (or bond) arises from the particular chemistry strategy chosen to link the amino acid ester motif R₁R₂R₃CNH— to the ring system A. Clearly the chemistry strategy for that coupling may vary widely, and thus many combinations of the variables Y, L¹, X¹ and z are possible. The precise combination of variables making up the linking chemistry between the amino acid ester motif and the ring system A will often be irrelevant to the primary binding mode of the compound as a whole. On the other hand, that linkage chemistry will in some cases pick up additional binding interactions with the enzyme.

It should also be noted that the benefits of the amino acid ester motif (facile entry into the cell, esterase hydrolysis within the cell, and accumulation within the cell of active carboxylic acid hydrolysis product) are best achieved when the linkage between the amino acid ester motif and the ring system A is not a substrate for peptidase activity within the cell, which might result in cleavage of the amino acid from the molecule. Of course, stability to intracellular peptidases is easily tested by incubating the compound with disrupted cell contents, and analysing for any such cleavage.

With the foregoing general observations in mind, taking the variables making up the radical —Y-L¹-X¹—[CH₂]_{z}— in turn:
z may be 0 or 1, so that a methylene radical linked to the ring system A is optional;
specific preferred examples of Y when macrophage selectivity is not required include —(C═O)—, —(C═O)NH—, and —(C═O)O—; Where macrophage selectivity is required any of the other options for Y, including the case where Y is a bond, are appropriate.
In the radical L¹, examples of Alk¹ and Alk² radicals, when present, include —CH₂—, —CH₂CH₂—, —CH₂CH₂CH₂—, —CH₂CH₂CH₂CH₂—, —CH═CH—, —CH═CHCH₂—, —CH₂CH═CH—, CH₂CH═CHCH₂—C≡C—, —C≡CCH₂—, —CH₂C≡C—, and CH₂C≡CCH₂. Additional examples of Alk¹ and Alk² include —CH₂W—, —CH₂CH₂W—, —CH₂CH₂WCH₂—, —CH₂CH₂WCH(CH₃)—, —CH₂WCH₂CH₂—, —CH₂WCH₂CH₂WCH₂— and —WCH₂CH₂— where W is —O—, —S—, —NH—, —N(CH₃)—, or —CH₂CH₂N(CH₂CH₂OH)CH₂—. Further examples of Alk¹ and Alk² include divalent cyclopropyl, cyclopentyl and cyclohexyl radicals.
In L¹, when n is 0, the radical is a hydrocarbon chain (optionally substituted and perhaps having an ether, thioether or amino linkage). Presently it is preferred that there be no optional substituents in L¹. When both m and p are 0, L¹ is a divalent mono- or bicyclic carbocyclic or heterocyclic radical with 5-13 ring atoms (optionally substituted). When n is 1 and at least one of m and p is 1, L¹ is a divalent radical including a hydrocarbon chain or chains and a mono- or bicyclic carbocyclic or heterocyclic radical with 5-13 ring atoms (optionally substituted). When present, Q may be, for example, a divalent phenyl, naphthyl, cyclopropyl, cyclopentyl, or cyclohexyl radical, or a mono-, or bi-cyclic heterocyclic radical having 5 to 13 ring members, such as piperidinyl, piperazinyl, indolyl, pyridyl, thienyl, or pyrrolyl radical, but 1,4-phenylene is presently preferred.
Specifically, in some embodiments of the invention, L¹, m and p may be 0 with n being 1. In other embodiments, n and p may be 0 with m being 1. In further embodiments, m, n and p may be all 0. In still further embodiments m may be 0, n may be 1 with Q being a monocyclic heterocyclic radical, and p may be 0 or 1. Alk¹ and Alk², when present, may be selected from —CH₂—, —CH₂CH₂—, and —CH₂CH₂CH₂— and Q may be 1,4-phenylene.

Specific examples of the radical —Y-L¹-X¹—[CH₂]_{z}— include —C(═O)— and —C(═O)NH— as well as —(CH₂)ᵥ—, —(CH₂)ᵥO—, —C(═O)—(CH₂)ᵥ—, —C(═O)—(CH₂)ᵥO—, —C(═O)—NH—(CH₂)_{w}—, —C(═O)—NH—(CH₂)_{w}O—

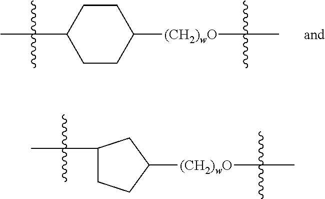

wherein v is 1, 2, 3 or 4 and w is 1, 2 or 3, such as —Y-L¹-X¹—[CH₂]_{z}—, is —CH₂—, —CH₂CH₂—, —CH₂CH₂CH₂—, —CH₂CH₂CH₂CH₂—, —CH₂O—, —CH₂CH₂O—, —CH₂CH₂CH₂O—, —CH₂CH₂CH₂CH₂O—, —C(═O)—CH₂—, —C(═O)—CH₂O—, —C(═O)—NH—CH₂—, or —C(═O)—NH—CH₂O—.

### The Group R₁

In one class of compounds of the invention, R₁ is a carboxylic acid group. Although compounds of this class may be administered as the carboxylic acid or a salt thereof, it is preferred that they be generated in the cell by the action of an intracellular esterase on a corresponding compound in which R₁ is an ester group.

The ester group R₁ must be one which in the compound of the invention is hydrolysable by one or more intracellular carboxylesterase enzymes to a carboxylic acid group. Intracellular carboxylesterase enzymes capable of hydrolysing the ester group of a compound of the invention to the corresponding acid include the three known human enzyme isotypes hCE-1, hCE-2 and hCE-3. Although these are considered to be the main enzymes, other enzymes such as biphenylhydrolase (BPH) may also have a role in hydrolysing the ester. In general, if the carboxylesterase hydrolyses the free amino acid ester to the parent acid it will also hydrolyse the ester motif when covalently conjugated to the inhibitor. Hence, the broken cell assay and/or the isolated carboxylesterase assay described herein provide a straightforward, quick and simple first screen for esters which have the required hydrolysis profile. Ester motifs selected in that way may then be re-assayed in the same carboxylesterase assay when conjugated to the inhibitor via the chosen conjugation chemistry, to confirm that it is still a carboxylesterase substrate in that background.

Subject to the requirement that they be hydrolysable by intracellular carboxylesterase enzymes, examples of particular ester groups R₁ include those of formula —(C═O)OR₁₄ wherein R₁₄ is R₉R₉R₁₀C— wherein
(i) R₈ is hydrogen, fluorine or optionally substituted (C₁-C₃)alkyl-(Z¹)ₐ—[(C₁-C₃)alkyl]_{b}- or (C₂-C₃)alkenyl-(Z¹)ₐ—[(C₁-C₃)alkyl]_{b}- wherein a and b are independently 0 or 1 and Z¹ is —O—, —S—, or —NR₁₁— wherein R₁₁ is hydrogen or (C₁-C₃)alkyl; and R₉ and R₁₀ are independently hydrogen or (C₁-C₃)alkyl-;
(ii) R₈ is hydrogen or optionally substituted R₁₂R₁₃N—(C₁-C₃)alkyl- wherein R₁₂ is hydrogen or (C₁-C₃)alkyl and R₁₃ is hydrogen or (C₁-C₃)alkyl; or R₁₂ and R₁₃ together with the nitrogen to which they are attached form an optionally substituted monocyclic heterocyclic ring of 5- or 6-ring atoms or bicyclic heterocyclic ring system of 8 to 10 ring atoms, and R₉ and R₁₀ are independently hydrogen or (C₁-C₃)alkyl-; or
(iii) R₈ and R₉ taken together with the carbon to which they are attached form an optionally substituted monocyclic carbocyclic ring of from 3 to 7 ring atoms or bicyclic carbocyclic ring system of 8 to 10 ring atoms, and R₁₀ is hydrogen.

In cases (i), (ii) and (iii) above, “alkyl” includes fluoroalkyl.

Within these classes (i), (ii) and (iii), R₁₀ is often hydrogen. Specific examples of R₁₄ include methyl, trifluoromethyl, ethyl, n- or iso-propyl, n-, sec- or tert-butyl, cyclohexyl, allyl, phenyl, benzyl, 2-, 3- or 4-pyridylmethyl, N-methylpiperidin-4-yl, tetrahydrofuran-3-yl, methoxyethyl, indanyl, norbornyl, dimethylaminomethyl or morpholinoethyl. Currently preferred is where R₁₄ is cyclopentyl.

Macrophages are known to play a key role in inflammatory disorders through the release of cytokines in particular TNFα and IL-1 (van Roon et al, Arthritis and Rheumatism, 2003, 1229-1238). In rheumatoid arthritis they are major contributors to the maintenance of joint inflammation and joint destruction. Macrophages are also involved in tumour growth and development (Naldini and Carraro, Curr Drug Targets Inflamm Allergy, 2005, 3-8). Hence agents that selectively target macrophage cell proliferation could be of value in the treatment of cancer and autoimmune disease. Targeting specific cell types would be expected to lead to reduced side-effects. The inventors have discovered a method of targeting IKK inhibitors to macrophages and other cells derived from the myelo-monocytic lineage such as monocytes, osteoclasts and dendritic cells. This is based on the observation that the way in which the esterase motif is linked to the IKK kinase inhibitor determines whether it is hydrolysed, and hence whether or not it accumulates in different cell types. Specifically it has been found that macrophages and other cells derived from the myelo-monocytic lineage contain the human carboxylesterase hCE-1 whereas other cell types do not. In the general formula (IA) or (IB) when the nitrogen of the esterase motif R₁C(R₂)(R₃)NH— is not directly linked to a carbonyl (—C(═O)—), i.e. when Y is not a —C(═O), —C(═O)O— or —C(═O)NR₃— radical, the ester will only be hydrolysed by hCE-1 and hence the inhibitors will selectively accumulate in macrophage-related cells. Herein, unless “monocyte” or “monocytes” is specified, the term macrophage or macrophages will be used to denote macrophages (including tumour associated macrophages) and/or monocytes.

### Subtituents R₂ and R₃

The substituents R₂ and R₃ may be regarded as the α-substituents of an α,α-disubstituted glycine or an α,α-disubstituted glycine ester. These substituents may therefore be the side chains of a natural or non-natural alpha-amino acid other than glycine, and in such side chains any functional groups may be protected.

For example, examples of R₂ and R₃ include phenyl and groups of formula —CRₐR_{b}R_{c} in which:
each of Rₐ, R_{b} and R_{c} is independently hydrogen, (C₁-C₆)alkyl, (C₂-C₆)alkenyl, (C₂-C₆)alkynyl, phenyl(C₁-C₆)alkyl, (C₃-C₈)cycloalkyl; or
R_{c} is hydrogen and Rₐ and R_{b} are independently phenyl or heteroaryl such as pyridyl; or
R_{c} is hydrogen, (C₁-C₆)alkyl, (C₂-C₆)alkenyl, (C₂-C₆)alkynyl, phenyl(C₁-C₆)alkyl, or (C₃-C₈)cycloalkyl, and Rₐ and R_{b} together with the carbon atom to which they are attached form a 3 to 8 membered cycloalkyl or a 5- to 6-membered heterocyclic ring; or
Rₐ, R_{b} and R_{c} together with the carbon atom to which they are attached form a tricyclic ring (for example adamantyl); or
Rₐ and R_{b} are each independently (C₁-C₆)alkyl, (C₂-C₆)alkenyl, (C₂-C₆)alkynyl, phenyl(C₁-C₆)alkyl, or a group as defined for R_{c} below other than hydrogen, or Rₐ and R_{b} together with the carbon atom to which they are attached form a cycloalkyl or heterocyclic ring, and R_{c} is hydrogen, —OH, —SH, halogen, —CN, —CO₂H, (C₁-C₄)perfluoroalkyl, —CH₂OH, —O(C₁-C₆)alkyl, —O(C₂-C₆)alkenyl, —S(C₁-C₆)alkyl, —SO(C₁-C₆)alkyl, —SO₂(C₁-C₆) alkyl, —S(C₂-C₆)alkenyl, —SO(C₂-C₆)alkenyl, —SO₂(C₂-C₆)alkenyl or a group -Q-W wherein Q represents a bond or —O—, —S—, —SO— or —SO₂— and W represents a phenyl, phenylalkyl, (C₃-C₈)cycloalkyl, (C₃-C₈)cycloalkylalkyl, (C₄-C₈)cycloalkenyl, (C₄-C₈)cycloalkenylalkyl, heteroaryl or heteroarylalkyl group, which group W may optionally be substituted by one or more substituents independently selected from, hydroxyl, halogen, —CN, —CONH₂, —CONH(C₁-C₆)alkyl, —CONH(C₁-C₆alkyl)₂, —CHO, —CH₂OH, (C₁-C₄)perfluoroalkyl, —O(C₁-C₆)alkyl, —S(C₁-C₆)alkyl, —SO(C₁-C₆)alkyl, —SO₂(C₁-C₆)alkyl, —NO₂, —NH₂, —NH(C₁-C₆)alkyl, —N((C₁-C₆)alkyl)₂, —NHCO(C₁-C₆)alkyl, (C₁-C₆)alkyl, (C₂-C₆)alkenyl, (C₂-C₆)alkynyl, (C₃-C₈)cycloalkyl, (C₄-C₈)cycloalkenyl, phenyl or benzyl.

Alternatively, the substituents R₂ and R₃, taken together with the carbon to which they are attached, may form a 3-6 membered saturated spiro cycloalkyl ring, such as a cyclopropyl, cyclopentyl or cyclohexyl ring or spiro heterocyclyl ring such as a piperidin-4-yl ring.

In some cases, at least one of the substituents R₂ and R₃ is a C₁-C₆ alkyl substituent, for example methyl, ethyl, or n- or iso-propyl.

In some embodiments, one of the substituents R₂ and R₃ is a C₁-C₆ alkyl substituent, for example methyl, ethyl, or n- or iso-propyl, and the other is selected from the group consisting of methyl, ethyl, n- and iso-propyl, n-, sec- and tert-butyl, phenyl, benzyl, thienyl, cyclohexyl, and cyclohexylmethyl.

In a particular case, the substituents R₂ and R₃ are each methyl.

For compounds of the invention which are to be administered systemically, esters with a slow rate of carboxylesterase cleavage are preferred, since they are less susceptible to pre-systemic metabolism. Their ability to reach their target tissue intact is therefore increased, and the ester can be converted inside the cells of the target tissue into the acid product. However, for local administration, where the ester is either directly applied to the target tissue or directed there by, for example, inhalation, it will often be desirable that the ester has a rapid rate of esterase cleavage, to minimise systemic exposure and consequent unwanted side effects. In the compounds of this invention, if the carbon adjacent to the alpha carbon of the alpha amino acid ester is mono-substituted, ie R₂ and R₃ are —CH₂R^{z} (R^{z} being the mono-substituent) then the esters tend to be cleaved more rapidly than if that carbon is di- or tri-substituted, as in the case where R₂ and R₃ are, for example, phenyl or cyclohexyl, or together form a ring.

As mentioned above, the compounds with which the invention is concerned are inhibitors of IKK, especially IKK-β kinase activity, and are therefore of use in the treatment of diseases modulated by IKK activity and the NF-kB cascade. Such diseases include neoplastic/proliferative, immune and inflammatory disease. In particular, uses of the compounds include treatment of cancers such as hepatocellular cancer or melanoma, but also including bowel cancer, ovarian cancer, head and neck and cervical squamous cancers, gastric or lung cancers, anaplastic oligodendrogliomas, glioblastoma multiforme or medulloblastomas; and treatment of rheumatoid arthritis, psoriasis, inflammatory bowel disease, Crohn's disease, ulcerative colitis, chronic obstructive pulmonary disease, asthma, multiple sclerosis, diabetes, atopic dermatitis, graft versus host disease, systemic lupus erythematosus, metabolic disorders e.g. Type II diabetes mellitus or neurological disorders e.g. Alzheimers.

The compounds with which the invention is concerned may be prepared for administration by any route consistent with their pharmacokinetic properties. The orally administrable compositions may be in the form of tablets, capsules, powders, granules, lozenges, liquid or gel preparations, such as oral, topical, or sterile parenteral solutions or suspensions. Tablets and capsules for oral administration may be in unit dose presentation form, and may contain conventional excipients such as binding agents, for example syrup, acacia, gelatin, sorbitol, tragacanth, or polyvinyl-pyrrolidone; fillers for example lactose, sugar, maize-starch, calcium phosphate, sorbitol or glycine; tabletting lubricant, for example magnesium stearate, talc, polyethylene glycol or silica; disintegrants for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavouring or colouring agents.

For topical application to the skin, the drug may be made up into a cream, lotion or ointment. Cream or ointment formulations which may be used for the drug are conventional formulations well known in the art, for example as described in standard textbooks of pharmaceutics such as the British Pharmacopoeia.
The compounds of the invention may be administered in inhaled form. Aerosol generation can be carried out using, for example, pressure-driven jet atomizers or ultrasonic atomizers, preferably using propellant-driven metered aerosols or propellant-free administration of micronized active compounds from, for example, inhalation capsules or other “dry powder” delivery systems.
The active compounds may be dosed as described depending on the inhaler system used. In addition to the active compounds, the administration forms may additionally contain excipients, such as, for example, propellants (e.g. Frigen in the case of metered aerosols), surface-active substances, emulsifiers, stabilizers, preservatives, flavourings, fillers (e.g. lactose in the case of powder inhalers) or, if appropriate, further active compounds.
For the purposes of inhalation, a large number of systems are available with which aerosols of optimum particle size can be generated and administered, using an inhalation technique which is appropriate for the patient. In addition to the use of adaptors (spacers, expanders) and pear-shaped containers (e.g. Nebulator®, Volumatic®), and automatic devices emitting a puffer spray (Autohaler®), for metered aerosols, in particular in the case of powder inhalers, a number of technical solutions are available (e.g. Diskhaler®, Rotadisk®, Turbohaler® or the inhalers for example as described EP-A-0505321).

For topical application to the eye, the drug may be made up into a solution or suspension in a suitable sterile aqueous or non aqueous vehicle. Additives, for instance buffers such as sodium metabisulphite or disodium edeate; preservatives including bactericidal and fungicidal agents such as phenyl mercuric acetate or nitrate, benzalkonium chloride or chlorhexidine, and thickening agents such as hypromellose may also be included.

The active ingredient may also be administered parenterally in a sterile medium. Depending on the vehicle and concentration used, the drug can either be suspended or dissolved in the vehicle. Advantageously, adjuvants such as a local anaesthetic, preservative and buffering agents can be dissolved in the vehicle.

The compounds of the invention may be used in conjunction with a number of known pharmaceutically active substances. For example, the compounds of the invention may be used with cytotoxics, HDAC inhibitors, kinase inhibitors, aminopeptidase inhibitors, protease inhibitors, bcl-2 antagonists, inhibitors of mTor and monoclonal antibodies (for example those directed at growth factor receptors). Preferred cytotoxics include, for example, taxanes, platins, anti-metabolites such as 5-fluoracil, topoisomerase inhibitors and the like. The medicaments of the invention comprising amino acid derivatives of formula (IA) or (IB), tautomers thereof or pharmaceutically acceptable salts, N-oxides, hydrates or solvates thereof therefore typically further comprise a cytotoxic, an HDAC inhibitor, a kinase inhibitor, an aminopeptidase inhibitor and/or a monoclonal antibody.

Further, the present invention provides a pharmaceutical composition comprising:
(a) an amino acid derivative of formula (IA) or (IB), or a pharmaceutically acceptable salt, N-oxide, hydrate or solvate thereof;
(b) a cytotoxic agent, an HDAC inhibitor, a kinase inhibitor, an aminopeptidase inhibitor, a protease inhibitor, a bcl-2 antagonist, an inhibitor of mTor and/or a monoclonal antibody; and
(c) a pharmaceutically acceptable carrier or diluent.

Also provided is a product comprising:
(a) an amino acid derivative of formula (IA) or (IB), or a pharmaceutically acceptable salt, N-oxide, hydrate or solvate thereof; and
(b) a cytotoxic agent, an HDAC inhibitor, a kinase inhibitor, an aminopeptidase inhibitor, a protease inhibitor, a bcl-2 antagonist, an inhibitor of mTor and/or a monoclonal antibody,
   for the separate, simultaneous or sequential use in the treatment of the human or animal body.

### Synthesis

There are multiple synthetic strategies for the synthesis of the compounds (IA) or (IB) with which the present invention is concerned, but all rely on known chemistry, known to the synthetic organic chemist. Thus, compounds according to formula (I) can be synthesised according to procedures described in the standard literature and are well-known to those skilled in the art. Typical literature sources are “Advanced organic chemistry”, 4^{th} Edition (Wiley), J March, “Comprehensive Organic Transformation”, 2^{nd} Edition (Wiley), R. C. Larock, “Handbook of Heterocyclic Chemistry”, 2^{nd} Edition (Pergamon), A. R. Katritzky), review articles such as found in “Synthesis”, “Acc. Chem. Res.”, “Chem. Rev”, or primary literature sources identified by standard literature searches online or from secondary sources such as “Chemical Abstracts” or “Beilstein”.

The compounds of the invention may be prepared by a number of processes generally described below and more specifically in the Examples hereinafter. In the reactions described below, it may be necessary to protect reactive functional groups, for example hydroxyl, amino and carboxy groups, where these are desired in the final product, to avoid their unwanted participation in the reactions [see for example Greene, T. W., “Protecting Groups in Organic Synthesis”, John Wiley and Sons, 1999]. Conventional protecting groups may be used in conjunction with standard practice. In some instances deprotection may be the final step in the synthesis of a compound of general formula (IA) or (IB), and the processes according to the invention described herein after are understood to extend to such removal of protecting groups.

As mentioned above, the compounds with which the invention is concerned are inhibitors of the IkB family, namely IKK-α and IKK-β, and are therefore of use in the treatment of cell proliferative disease, such as cancer, and in treatment of inflammation, in humans and other mammals.

### Abbreviations

MeOH=methanol
EtOH=ethanol
EtOAc=ethyl acetate
DCM=dichloromethane
DIBAL=di-isobutylaluminium hydride
DMF=dimethylformamide
DME=1,2-dimethoxy ethane
DMSO=dimethyl sulfoxide
DMAP=4-dimethylamino pyridine
TFA=trifluoroacetic acid
THF=tetrahydrofuran
Na₂CO₃=sodium carbonate
HCl=hydrochloric acid
DIPEA=diisopropylethylamine
LiHMDS=lithium bis(trimethylsilyl)amide
MP-CNBH₃=macroporous triethylammonium methylpolystyrene cyanoborohydride
NaH=sodium hydride
NaOH=sodium hydroxide
NaHCO₃=sodium hydrogen carbonate
Pd/C=palladium on carbon
PdCl₂(dppf)=[1,1-Bis(diphenylphosphino)ferrocene]dichloropalladium(II).
EDCI=1-ethyl-3-(3-dimethylaminopropyl)carbodiimide
KOAc=potassium acetate
TBAI=tetrabutyl ammonium iodide
ml=millilitre(s)
g=gram(s)
mg=milligram(s)
mol=mole(s)
mmol=millimole(s)
Sat=saturated
LCMS=high performance liquid chromatography/mass spectrometry
NMR=nuclear magnetic resonance
RT=room temperature

Commercially available reagents and solvents (HPLC grade) were used without further purification. Solvents were removed using a Buchi rotary evaporator. Microwave irradiation was carried out using a CEM Discovery model set at 300 W. Purification of compounds by flash chromatography column was performed using silica gel, particle size 40-63μ μm (230-400 mesh) obtained from Fluorochem. Purification of compounds by preparative HPLC was performed on a Agilent prep system using reverse phase Agilent prep-C18 columns (5 μm, 50×21.2 mm), gradient 0-100% B (A=water/0.1% ammonia or 0.1% formic acid and B=acetonitrile/0.1% ammonia or 0.1% formic acid) over 10 min, flow=28 ml/min, UV detection at 254 nm.

¹H NMR spectra were recorded on a Bruker 400 or 300 MHz AV spectrometer in deuterated solvents. Chemical shifts (δ) are in parts per million. Thin-layer chromatography (TLC) analysis was performed with Kieselgel 60 F₂₅₄ (Merck) plates and visualized using UV light.

Analytical HPLC/MS were obtained as follows: Agilent Prep-C18 Scalar column, 5 μm (4.6×50 mm, flow rate 2.5 ml/min) eluting with a H₂O-MeCN gradient containing 0.1% v/v formic acid over 7 minutes with UV detection at 254 nm. Gradient information: 0.0-0.5 min: 95% H₂O-5% MeCN; 0.5-5.0 min; Ramp from 95% H₂O-5% MeCN to 5% H₂O-95% MeCN; 5.0-5.5 min: Hold at 5% H₂O-95% MeCN; 5.5-5.6 min: Hold at 5% H₂O-95% MeCN, flow rate increased to 3.5 ml/min; 5.6-6.6 min: Hold at 5% H₂O-95% MeCN, flow rate 3.5 ml/min; 6.6-6.75 min: Return to 95% H₂O-5% MeCN, flow rate 3.5 ml/min; 6.75-6.9 min: Hold at 95% H₂O-5% MeCN, flow rate 3.5 ml/min; 6.9-7.0 min: Hold at 95% H₂O-5% MeCN, flow rate reduced to 2.5 ml/min. Mass spectra were obtained using an Agilent multimode source in either the positive (APCI+ESI⁺) or negative (APCI+ESI⁻) mode.

Examples of such methods that may be employed to the synthesis of compounds of general formula (IA) and (IB) are set out, but not limited to the reactions shown in Schemes 1-5 below.

Scheme 1 illustrates the general synthetic route for the preparation of the examples described below, using traditional Suzuki chemistry to couple the relevant boronate ester (or acid) with the central thiophene core (Intermediate 1) to give the corresponding Intermediate 2.

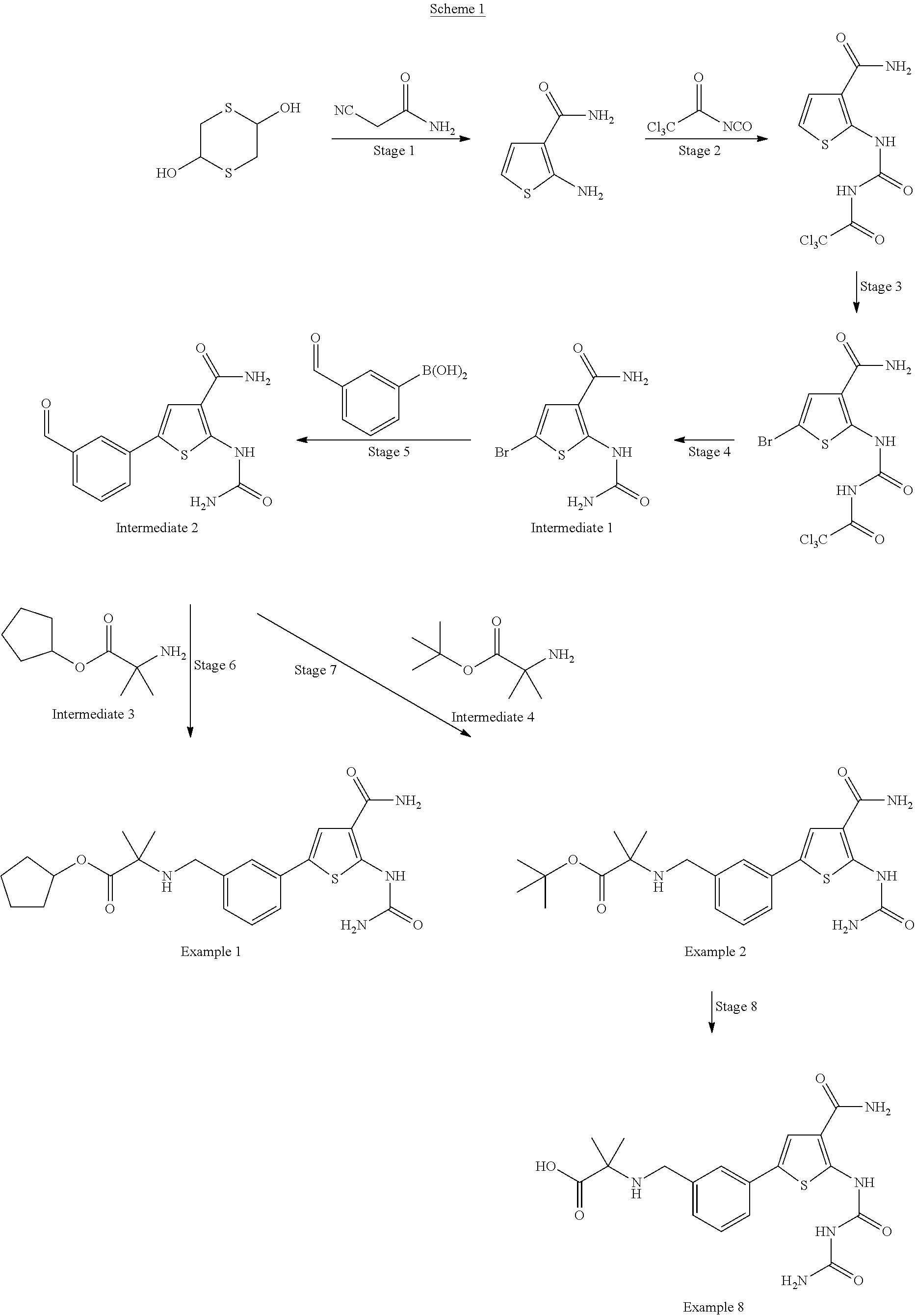

Scheme 2 illustrates the synthetic route for the preparation of Example 3 using Suzuki chemistry to couple Intermediate 6 with the central thiophene core (Intermediate 1).

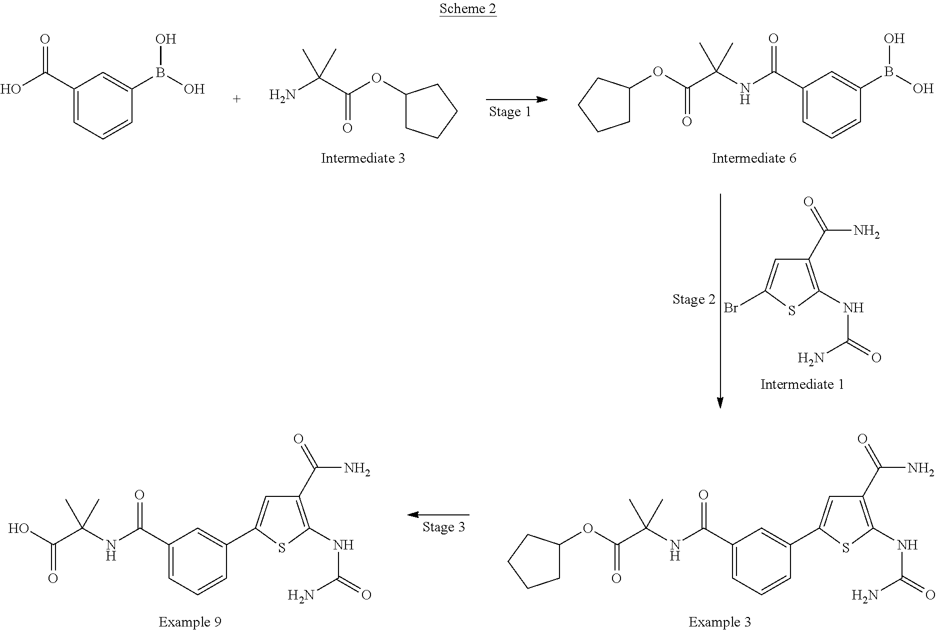

Scheme 3 describes the synthetic route followed for the preparation of Example 4.

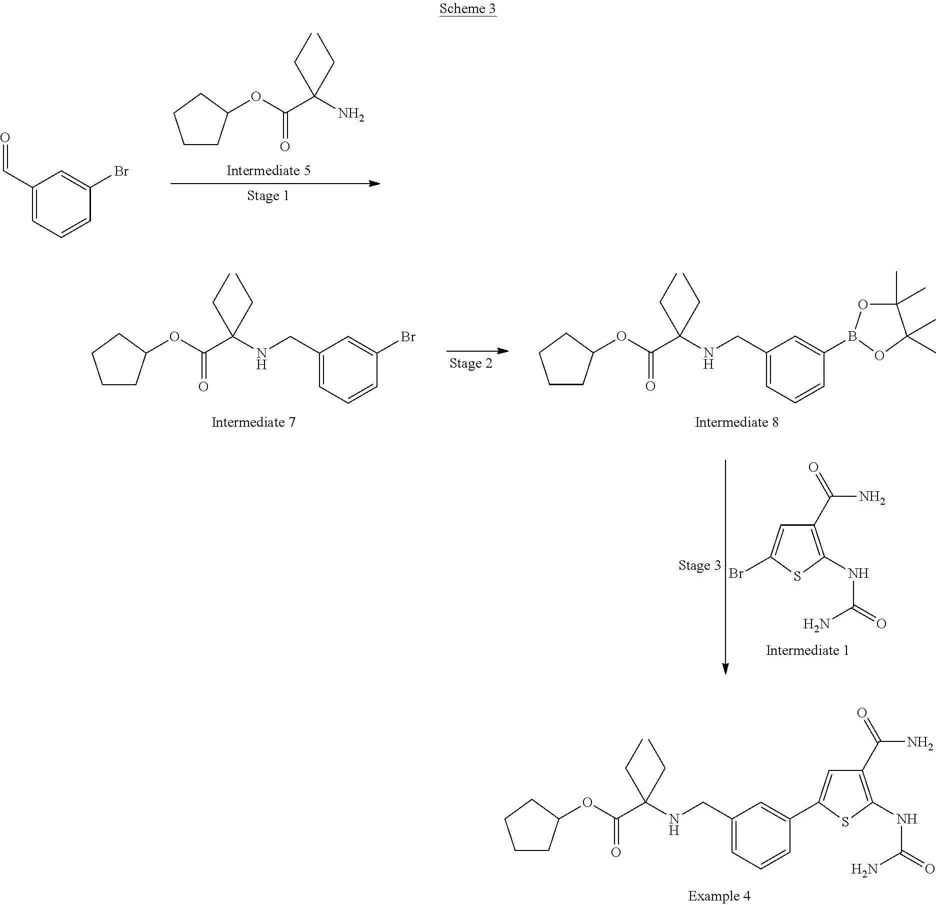

Scheme 4 describes the synthetic route followed for the preparation of Example 6 and Example 10.

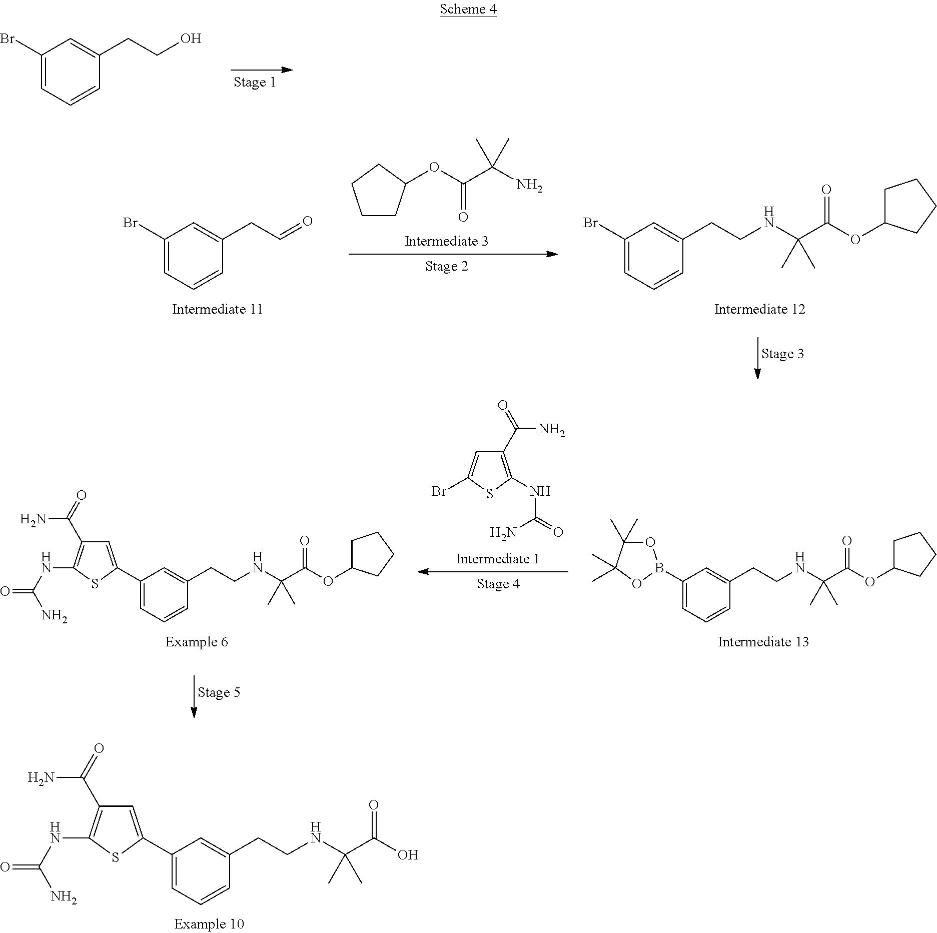

Scheme 5 describes the synthetic route followed for the preparation of Example 7 and Example 11.

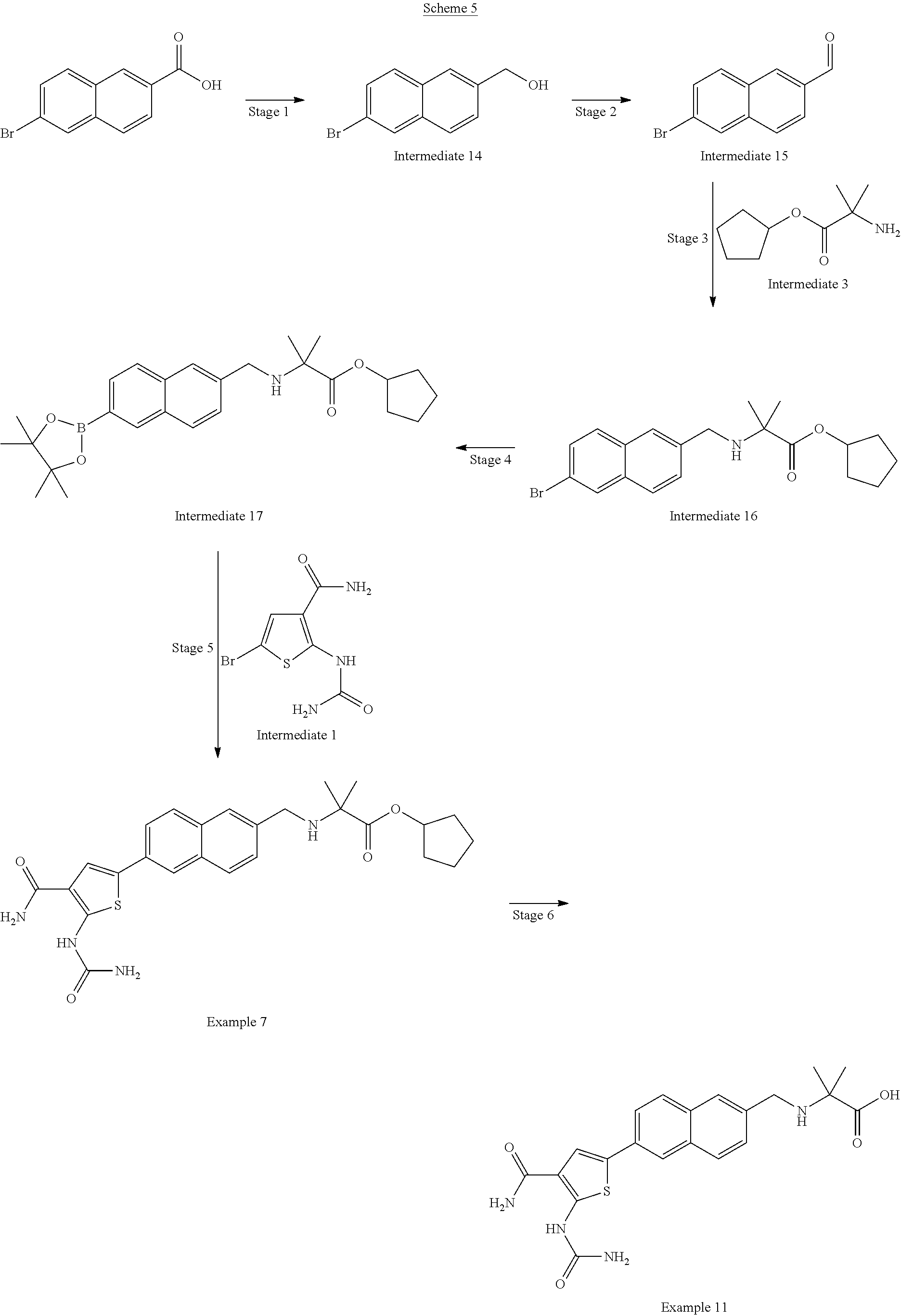

### Intermediates

### Intermediate 1: 5-Bromo-2-(carbamoylamino)thiophene-3-carboxamide

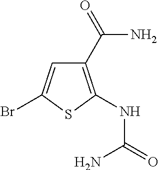

The synthesis of Intermediate 1 described by Stages 1-4 in Scheme 1 is detailed within WO03104218.

### Intermediate 2: 2-(Carbamoylamino)-5-(3-formylphenyl)thiophene-3-carboxamide

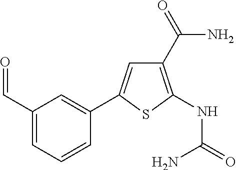

To a mixture of 5-bromo-2-(carbamoylamino)thiophene-3-carboxamide (1.0 g, 3.79 mmol), 3-formylphenylboronic acid (0.625 g, 4.17 mmol) and tetrakis(triphenylphosphine) palladium (0.438 g, 0.379 mmol) in DME (50 ml) was added a saturated aqueous solution of sodium hydrogen carbonate (10 ml). The reaction vessel was flushed with nitrogen and heated to 90° C. overnight. The reaction mixture was concentrated under reduced pressure using a rotary evaporator. The resultant dark brown residue was dissolved in DCM (17 ml) and stirred with aqueous 2M sodium hydroxide solution (8.5 ml) for 20 minutes. Diethyl ether (20 ml) was added and the mixture stirred for a further 30 minutes. The resultant suspension was sonicated for 2 minutes. Filtration gave a precipitate, which was triturated with hot diethyl ether to give a coloured solid (440 mg).

LCMS: m/z 288 [M−H]⁺, m/z 290 [M+H]⁺.

### Intermediate 3: Cyclopentyl 2-methylalaninate hydrochloride

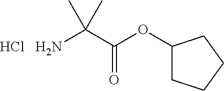

Intermediate 3 was synthesised using the route shown in Scheme 6 below.

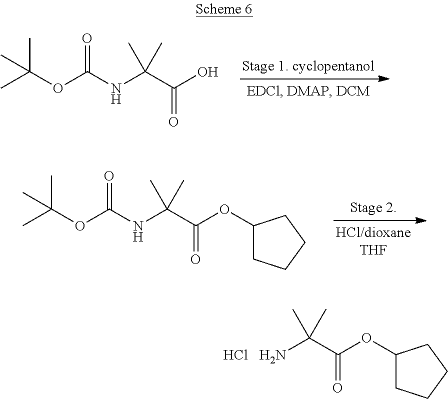

### Stage 1—Cyclopentyl N-(tert-butoxycarbonyl)-2-methylalaninate

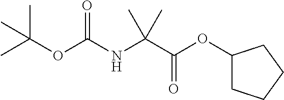

To a solution of N-(tert-butoxycarbonyl)-2-methylalanine (1.00 g, 4.92 mmol) in DCM (10 ml) at 0° C. was added cyclopentanol (0.83 ml, 9.84 mmol), EDCI (1.06 g, 5.42 mmol) and finally DMAP (60 mg, 0.49 mmol). The reaction mixture was warmed to RT and stirred for 18 hours. The DCM was removed in vacuo to give a clear oil. The crude residue was dissolved in EtOAc (100 ml) and washed with water, 1M NaHCO₃ and brine. The organic phase was dried (MgSO₄) and concentrated in vacuo. The crude extract was purified by column chromatography (10% EtOAc in heptane) to yield the desired product as a clear oil (0.254 g, 20%).

¹H NMR (300 MHz, CDCl₃) δ: 5.25-5.17 (1H, m), 5.04 (1H, br s), 1.93-1.54 (8H, m), 1.49 (6H, s), 1.45 (9H, s).

### Stage 2—Cyclopentyl 2-methylalaninate hydrochloride (Intermediate 3)

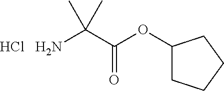

Cyclopentyl N-(tert-butoxycarbonyl)-2-methylalaninate (0.254 g, 0.93 mmol) was dissolved in THF (5 ml) and treated with 4M HCl/dioxane (2 ml) and the reaction mixture was stirred at RT for 24 hours. The crude mixture was concentrated under reduced pressure and triturated with Et₂O to give a white precipitate. This was further washed with Et₂O to give the desired product as a white powder (0.16 g, 82%).

¹H NMR (300 MHz, DMSO-d₆) δ: 8.58 (3H, br s), 5.21-5.14 (1H, m), 1.93-1.78 (2H, m), 1.74-1.53 (6H, m), 1.45 (6H, s).

### Intermediate 4: tert-Butyl 2-methylalaninate

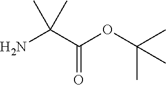

Intermediate 4 was synthesised using the route shown in Scheme 7 below.

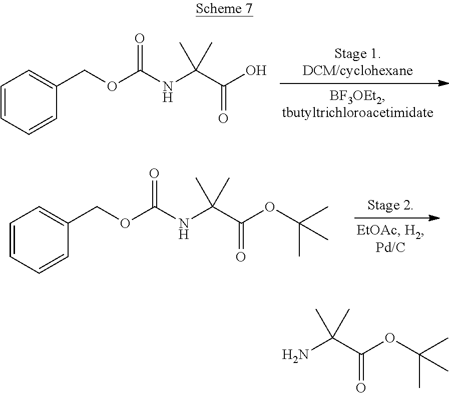

### Stage 1—tert-Butyl N-[(benzyloxy)carbonyl]-2-methylalaninate

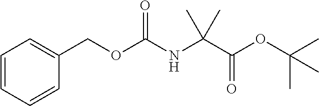

To a solution of N-[(benzyloxy)carbonyl]-2-methylalanine (1 g, 4.21 mmol) in DCM (10 ml anhydrous), cyclohexane (10 ml) at 0° C. under nitrogen was added boron trifluoride diethyl etherate (7 μl, catalytic). tert-Butyl 2,2,2-trichloroacetimidate (1.51 ml, 8.43 mmol) in cyclohexane (10 ml) was then added slowly over 30 minutes before allowing to warm to RT. Reaction was allowed to stir at RT for 16 hours. To the crude reaction mixture was added 190 mg of NaHCO₃ and the reaction filtered. The mother liquors were concentrated in vacuo. The crude extract was purified by column chromatography (10% EtOAc in heptane) to yield the desired product (0.863 g, 70%).

¹H NMR (300 MHz, CDCl₃) δ: 7.39-7.31 (5H, m), 5.46 (1H, br s), 5.10 (2H, s), 1.54 (6H, s), 1.45 (9H, s).

### Stage 2—tert-butyl 2-methylalaninate (Intermediate 4)

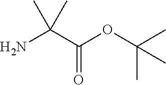

To a solution of tert-Butyl N-[(benzyloxy)carbonyl]-2-methylalaninate (0.86 mg, 2.90 mmol) in EtOAc (20 ml) was added 100 mg of 10% palladium on carbon catalyst. The mixture was evacuated and stirred under an atmosphere of hydrogen for 18 hours, filtered through Celite®, washed with EtOAc and concentrated in vacuo. The product was isolated as a yellow oil (0.45 mg, 96%) which contained traces of EtOAc.

¹H NMR (300 MHz, CDCl₃) δ: 1.48 (9H, s), 1.32 (6H, s).

### Intermediate 5: Cyclopentyl 2-amino-2-ethylbutanoate hydrochloride

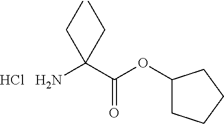

Intermediate 5 was prepared in a similar manner to Intermediate 3 using the synthetic route described in Scheme 6.

LCMS: m/z 200 [M+H]⁺.

### Intermediate 6: Cyclopentyl N-[3-(dihydroxyboryl)benzoyl]-2-methylalaninate

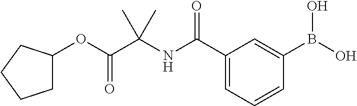

To a solution of 3-carboxy benzene boronic acid (200 mg, 1.2 mmol) in anhydrous DCM (15 ml) at 0° C. were added HOBt (162 mg, 1.2 mmol), EDCI (230 mg, 1.2 mmol) and the mixture stirred at 0° C. for 20 min. A solution of Intermediate 3 (310 mg, 1.81 mmol) in DCM (5 ml) was added and the mixture was stirred at RT for 4 hours. The reaction was diluted with DCM (10 ml) and washed with 1M aqueous HCl, 1M aqueous Na₂CO₃ and brine. The organic phase was dried (MgSO₄) and concentrated under reduced pressure to yield the desired product as a white solid (198 mg, 90%).

LCMS: m/z 320 [M+H]⁺.

### Intermediate 7: Cyclopentyl 2-[(3-bromobenzyl)amino]-2-ethylbutanoate

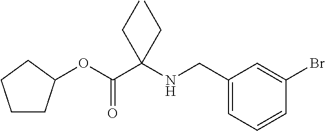

To a solution of 3-bromobenzaldehyde (0.49 g, 2.64 mmol) in anhydrous DCM (10 ml) under nitrogen was added Intermediate 5 (0.75 g, 3.17 mmol) and the mixture left to stir for 20 minutes before the addition of sodium triacetoxyborohydride (1.68 g, 7.93 mmol). The reaction was stirred at room temperature overnight then quenched with water (40 ml). The layers were separated, the aqueous layer was extracted with DCM and the combined organic layers were dried (MgSO₄), filtered and evaporated to dryness to give the crude product. Purification by column chromatography (50% EtOAc in heptane) afforded the title compound as a colourless oil (0.5 g, 52% yield).

LCMS: m/z 369 [M+H]⁺.

### Intermediate 8: Cyclopentyl 2-ethyl-2-{[3-(4,4,5,5-tetramethyl-1,3,2-dioxaborolan-2-yl)benzyl]amino}butanoate

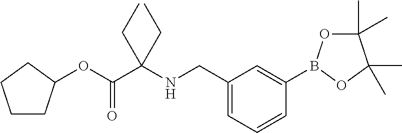

Cyclopentyl 2-[(3-bromobenzyl)amino]-2-ethylbutanoate (Intermediate 7) (0.5 g, 1.37 mmol) was dissolved in DMSO (10 ml) under nitrogen atmosphere and bis(pinacolato)diboron (0.52 g, 2.06 mmol) was added followed by PdCl₂(dppf) (0.056 g, 0.07 mmol) and potassium acetate (0.2 g, 2.06 mmol). The mixture was heated at 65° C. for 3 hours. The reaction was cooled to RT and partitioned between EtOAc (20 ml) and water (20 ml). The aqueous layer was extracted with EtOAc and the combined organic extracts were washed with brine, dried over MgSO₄ and concentrated in vacuo to leave a brown oil. Purification by column chromatography (50% EtOAc in heptane) afforded the title compound as a yellow oil (0.32 g, 58% yield).

LCMS: m/z 416 [M+H]⁺.

### Intermediate 9: Cyclopentyl N-(4-bromobenzyl)-2-methylalaninate

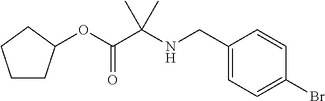

Intermediate 9 was prepared in a similar manner to Intermediate 7 using 4-bromobenzaldehyde and Intermediate 3.

LCMS: m/z 341 [M+H]⁺.

### Intermediate 10: Cyclopentyl 2-methyl-N-[4-(4,4,5,5-tetramethyl-1,3,2-dioxaborolan-2-yl)benzyl]alaninate

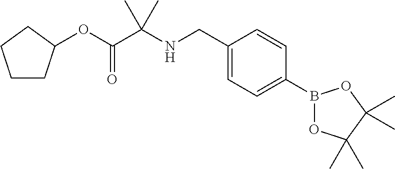

Intermediate 10 was prepared following a similar procedure to Intermediate 8 using Intermediate 9.

LCMS: m/z 388 [M+H]⁺.

### Intermediate 11: (3-bromophenyl)acetaldehyde

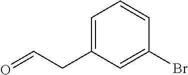

To a solution of 2-(3-bromophenyl)ethanol (4.9 g, 24.4 mmol) in DCM (20 ml) at 0° C. was added Dess Martin periodinane (10.8 g, 25 mmol) and the mixture was warmed to RT and stirred overnight. The reaction was diluted with DCM (100 ml) and stirred with a saturated solution of sodium thiosulphate (100 ml) and saturated NaHCO₃ (100 ml). The layers were separated and the organic layer was dried (MgSO₄), filtered and evaporated under reduced pressure to leave the crude product as an orange oil (4.07 g, 84%). The product was used without purification.

LCMS: m/z 200 [M+H]⁺.

### Intermediate 12: Cyclopentyl N-[2-(3-bromophenyl)ethyl]-2-methylalaninate

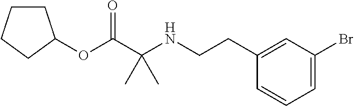

Intermediate 12 was prepared from Intermediates 3 and 11. following a similar procedure to Intermediate 7.

LCMS: m/z 355 [M+H]⁺.

### Intermediate 13: Cyclopentyl 2-methyl-N-{2-[3-(4,4,5,5-tetramethyl-1,3,2-dioxaborolan-2-yl)phenyl]ethyl}alaninate

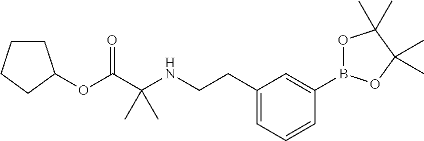

Intermediate 13 was prepared from Intermediate 12. following a similar procedure to Intermediate 8.

LCMS: m/z 424 [M+Na]

### Intermediate 14: (6-bromo-2-naphthyl)methanol

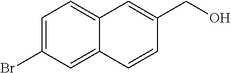

To a solution of 6-bromo-2-naphthoic acid (1 g, 3.98 mmol) in THF (20 ml) at 0° C. was added 2M borane dimethyl sulfide in THF (0.53 ml, 5.97 mmol) portion wise. The mixture was warmed to RT and stirred overnight. The reaction was cooled to 0° C. and MeOH was added. The solution was concentrated under reduced pressure. The crude product was purified by column chromatography (EtOAc in heptane) to afford the title compound (0.4 g, 89%).

LCMS: m/z 238 [M+H]⁺.

### Intermediate 15: 6-bromo-2-naphthaldehyde

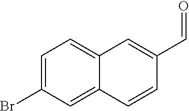

To a solution of (6-bromo-2-naphthyl)methanol (Intermediate 14) (0.84 g, 3.56 mmol) was added manganese oxide (2.29 g, 26.45 mmol) and the suspension stirred at RT for 48 hours. The reaction mixture was filtered through a pad of celite and concentrated under reduced pressure. The product was used without purification. (0.8 g, 95%).

LCMS: m/z 236 [M+H]⁺.

### Intermediate 16: Cyclopentyl N-[(6-bromo-2-naphthyl)methyl]-2-methylalaninate

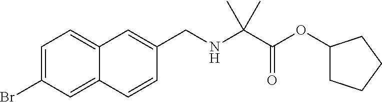

Intermediate 16 was prepared from Intermediates 3 and 15. following a similar procedure to Intermediate 7.

LCMS: m/z 391 [M+H]⁺.

### Intermediate 17: Cyclopentyl 2-methyl-N-{[6-(4,4,5,5-tetramethyl-1,3,2-dioxaborolan-2-yl)-2-naphthyl]methyl}alaninate

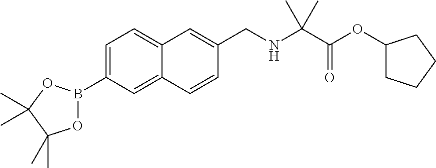

Intermediate 17 was prepared from Intermediate 16. following a similar procedure to Intermediate 8.

LCMS: m/z 438 [M+H]⁺.

### EXAMPLES

The following examples illustrate the preparation of the specific compounds of the invention, and the IKK inhibitory properties thereof:

### Example 1

### Cyclopentyl N-{3-[4-carbamoyl-5-(carbamoylamino)-2-thienyl]benzyl}-2-methylalaninate

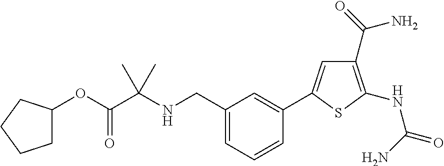

To a solution of 2-(carbamoylamino)-5-(3-formylphenyl)thiophene-3-carboxamide (Intermediate 2) (0.24 g, 0.83 mmol) in anhydrous tetrahydrofuran (8 ml) under nitrogen was added Intermediate 3 (0.197 g, 1.24 mmol) and the mixture left to stir for 20 minutes before the addition of sodium triacetoxyborohydride (0.528 g, 2.49 mmol). The reaction was stirred at room temperature overnight. The reaction was quenched with water. Tetrahydrofuran was removed under reduced pressure and the product extracted with dichloromethane (2×20 ml). The organic layers were combined, dried (MgSO₄), filtered and evaporated to dryness to give the crude product. Purification by preparative HPLC afforded the title compound (50 mg).

¹H NMR (300 MHz, CD₃OD) δ 7.74-7.67 (2H, m), 7.61 (1H, s), 7.53-7.44 (1H, m), 7.42-7.36 (1H, m), 5.40-5.32 (1H, m), 4.23 (2H, s), 2.02-1.69 (8H, m), 1.67 (6H, s).

LCMS: m/z 445 [M+H]⁺.

The following example was prepared in a similar manner to Example 1.

### Example 2

### tert-Butyl N-{3-[4-carbamoyl-5-(carbamoylamino)-2-thienyl]benzyl}-2-methylalaninate

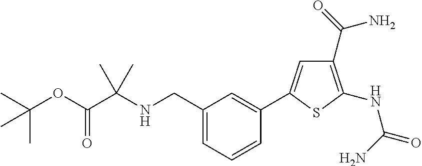

From Intermediate 2 and Intermediate 4.

¹H NMR (300 MHz, CD₃OD) δ 7.75-7.68 (2H, m), 7.62 (1H, s), 7.50 (1H, t, J=7.6 Hz), 7.42-7.38 (1H, m), 4.22 (2H, s), 1.66 (6H, s), 1.59 (9H, s).

LCMS: m/z 433 [M+H]⁺.

### Example 3

### Cyclopentyl N-{3-[4-carbamoyl-5-(carbamoylamino)-2-thienyl]benzoyl}-2-methylalaninate

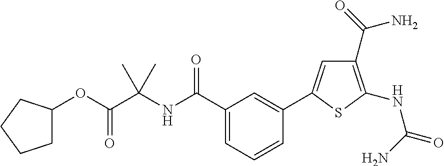

To a solution of Intermediate 6 (198 mg, 0.62 mmol) in DME (4 ml), was added Intermediate 1 (136 mg, 0.51 mmol) and tetrakis(triphenylphosphine) palladium (0.06 g). 2 ml of saturated aqueous NaHCO₃ was then added. The suspension was degassed with nitrogen and heated at reflux for 16 hours. The reaction was cooled to RT, poured in water (5 ml), extracted with EtOAc (2×20 ml). The combined organic layers were washed with brine, dried (MgSO₄) and concentrated under reduced pressure to afford the crude product. Purification by column chromatography (4% MeOH in DCM) gave the title compound as a light orange solid (195 mg, 25%).

¹H NMR (300 MHz, CD₃OD) δ 7.97 (3H, t, J=1.5 Hz), 7.74-7.69 (1H, m), 7.67-7.60 (2H, m), 7.44 (1H, t, J=7.8 Hz), 5.21-5.14 (1H, m), 1.89-1.58 (8H, m), 1.55 (6H, s).

LCMS: m/z 459 [M+H]⁺.

### Example 4

### Cyclopentyl 2-({3-[4-carbamoyl-5-(carbamoylamino)-2-thienyl]benzyl}amino)-2-ethylbutanoate

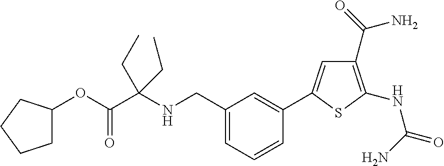

To a solution of Intermediate 1 (0.19 g, 0.73 mmol) in DME (10 ml) under nitrogen was added cyclopentyl 2-ethyl-2-{[3-(4,4,5,5-tetramethyl-1,3,2-dioxaborolan-2-yl)benzyl]amino}butanoate (Intermediate 8) (0.33 g, 0.8 mmol) followed by Pd(PPh₃)₄ (0.083 g, 0.07 mmol) and saturated aqueous NaHCO₃ (1 ml). The reaction mixture was stirred at 80° C. overnight then cooled to RT, partitioned between EtOAc (40 ml) and water (40 ml). The aqueous layer was extracted with EtOAc and the combined organic extracts were washed with brine, dried over MgSO₄ and concentrated in vacuo. Purification by preparative HPLC afforded the title compound as a yellow oil (0.015 g, 4% yield).

¹H NMR (300 MHz, DMSO-d₆) δ: 11.04 (1H, s), 9.28 (2H, br s) 7.81-7.68 (3H, m), (7.49-7.38 (3H, m), 6.99 (1H, br s), 5.26 (1H, m), 4.12 (2H, m), 2.01-1.91 (6H, m), 1.71-1.66 (6H, m), 0.94 (6H, t, J=7.3 Hz). LCMS: m/z 473 [M+H]⁺.

Examples 5-7 were prepared in a similar manner to Example 4.

### Example 5

### Cyclopentyl N-{4-[4-carbamoyl-5-(carbamoylamino)-2-thienyl]benzyl}-2-methylalaninate

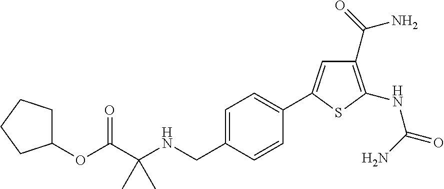

From Intermediate 10 and Intermediate 1.

¹H NMR (300 MHz, DMSO-d₆) δ: 11.00 (1H, s), 7.84 (2H, br s), 7.47 (2H, d, J=8.1 Hz), 7.34 (3H, d, J=8.1 Hz), 6.99 (2H, br s), 5.13-5.06 (1H, m), 3.59 (2H, br s), 1.91-1.77 (2H, m), 1.73-1.53 (6H, m), 1.26 (6H, s). LCMS: m/z 445 [M+H]⁺.

### Example 6

### Cyclopentyl N-(2-{3-[4-carbamoyl-5-(carbamoylamino)-2-thienyl]phenyl}ethyl)-2-methylalaninate

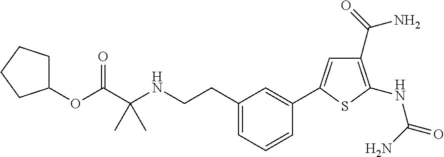

From Intermediate 1 and Intermediate 13.

¹H NMR (300 MHz, DMSO-d₆) δ 11.00 (1H, s), 7.72 (1H, s), 7.4 (1H, br s), 7.27-7.38 (3H, m), 7.07 (1H, d, J=7.3 Hz), 6.95 (2H, br s), 5.01 (1H, s), 2.68 (4H, d, J=5.3 Hz), 1.75 (2H, br. s.), 1.54 (6H, m), 1.16 (6H, s). LCMS: m/z 459 [M+H]⁺.

### Example 7

### Cyclopentyl N-({6-[4-carbamoyl-5-(carbamoylamino)-2-thienyl]-2-naphthyl}methyl)-2-methylalaninate

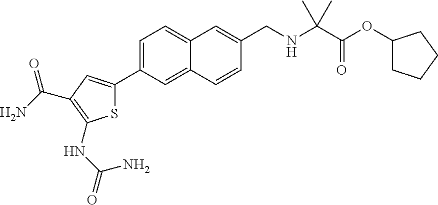

From Intermediate 1 and Intermediate 17.

¹H NMR (300 MHz, DMSO-d₆) δ 11.04 (1H, br s), 7.98-7.94 (1H, m), 7.92-7.85 (3H, m), 7.80-7.68 (3H, m), 7.51-7.45 (1H, m), 7.35 (1H, br s), 7.02 (2H, br s), 5.14-5.07 (1H, m), 3.75 (2H, m), 1.93-1.77 (2H, m), 1.74-1.52 (6H, m), 1.28 (6H, s). LCMS: m/z 495 [M+H]⁺.

### Example 8

### N-{3-[4-Carbamoyl-5-(carbamoylamino)-2-thienyl]benzyl}-2-methylalanine

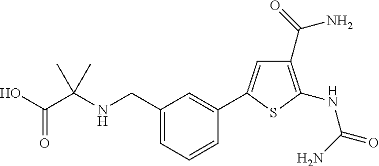

From Example 2.

To a solution of tert-butyl N-{3-[4-carbamoyl-5-(carbamoylamino)-2-thienyl]benzyl}-2-methylalaninate (Example 2) (30 mg, 0.07 mmol) in dichloromethane (1 ml) was added trifluoroacetic acid (1 ml). The reaction was stirred at room temperature overnight. The solvent was removed under reduced pressure and the residue was triturated in diethyl ether. The resulting solid was collected by filtration and dried under reduced pressure. Purification by preparative HPLC gave the title compound (17 mg, 75%).

¹H NMR (300 MHz, CD₃OD) δ 7.74-7.68 (2H, m), 7.63-7.60 (1H, m), 7.52-7.44 (1H, m), 7.42-7.37 (1H, m), 4.24 (2H, s), 1.70 (6H, s). LCMS: m/z 377 [M+H]⁺.

### Example 9

### N-{3-[4-Carbamoyl-5-(carbamoylamino)-2-thienyl]benzoyl}-2-methylalanine

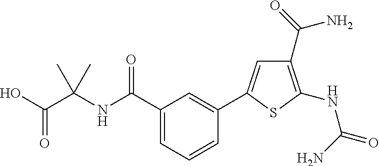

From Example 3.

To a solution of cyclopentyl N-{3-[4-carbamoyl-5-(carbamoylamino)-2-thienyl]benzoyl}-2-methylalaninate (Example 3) (194 mg, 0.42 mmol) in tetrahydrofuran (5 ml) was added a solution of lithium hydroxide (50 mg, 2.11 mmol) in water (5 ml). The reaction was stirred at 40° C. overnight. The solvent was removed in vacuo and to the residue was added water (2 ml). The pH was adjusted to pH 5 using 1M HCl. The precipitate was collected by filtration and washed sequentially with water and diethyl ether before drying under reduced pressure. The crude product was purified by preparative HPLC to afford the title compound (33 mg, 20%).

¹H NMR (300 MHz, CD₃OD) δ 8.02-7.99 (2H, m), 7.79-7.64 (2H, m), 7.63 (1H, s), 7.45 (1H, t, J=7.8 Hz), 1.60 (6H, s). LCMS: m/z 781 [2M+H]⁺.

Examples 10 and 11 were prepared following the same procedure as for Example 8.

### Example 10

### N-(2-{3-[4-carbamoyl-5-(carbamoylamino)-2-thienyl]phenyl}ethyl)-2-methylalanine

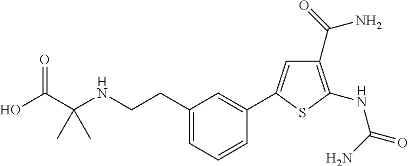

From Example 6.

¹H NMR (300 MHz, DMSO-d₆) δ 11.0 (1H, s), 9.0 (1H, br s), 7.75 (1H, s), 7.65 (1H, s), 7.45 (6H, m), 7.15 (1H, m), 6.96 (2H, m), 3.41 (4H, m), 1.50 (6H, s). LCMS: m/z 391 [M+H]⁺.

### Example 11

### N-({6-[4-carbamoyl-5-(carbamoylamino)-2-thienyl]-2-naphthyl}methyl)-2-methylalanine

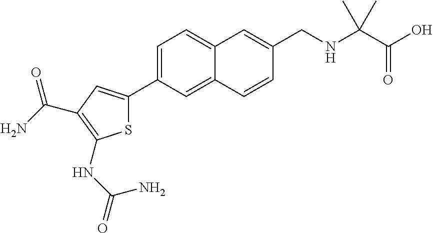

From Example 7.

¹H NMR (300 MHz, DMSO-d₆) δ 11.06 (1H, br s), 8.02-7.87 (5H, m), 7.80-7.71 (2H, m), 7.63-7.56 (1H, m), 7.37 (1H, br s), 7.03 (2H, br s), 4.07 (2H, s), 1.39 (6H, s).

### Measurement of Biological Activity

### IKKβ Enzyme Assay

The ability of compounds to inhibit IKK-β kinase activity was measured in an assay performed by Invitrogen (Paisley, UK). The Z′-LYTE™ biochemical assay employs a fluorescence-based, coupled-enzyme format and is based on the differential sensitivity of phosphorylated and non-phosphorylated peptides to proteolytic cleavage. The peptide substrate is labelled with two fluorophores—one at each end—that make up a FRET pair. In the primary reaction, the kinase transfers the gamma-phosphate of ATP to a single serine or threonine residue in a synthetic FRET-peptide. In the secondary reaction, a site-specific protease recognizes and cleaves non-phosphorylated FRET-peptides. Phosphorylation of FRET-peptides suppresses cleavage by the Development Reagent. Cleavage disrupts FRET between the donor (i.e. coumarin) and acceptor (i.e. fluorescein) fluorophores on the FRET-peptide, whereas uncleaved, phosphorylated FRET-peptides maintain FRET. A radiometric method, which calculates the ratio (the Emission Ratio) of donor emission to acceptor emission after excitation of the donor fluorophore at 400 nm, is used to quantitate reaction progress.

The final 10 μL Kinase Reaction consists of 0.9-8.0 ng IKBKB (IKK-β), 2 μM Ser/Thr 05 Peptide and ATP in 50 mM HEPES pH 7.5, 0.01% BRIJ-35, 10 mM MgCl₂, 1 mM EGTA. The assay is performed at an ATP concentration at, or close to the Km. After the 60 minute Kinase Reaction incubation at room temperature, 5 μL of a 1:128 dilution of Development Reagent is added. The assay plate is incubated for a further 60 minutes at room temperature and read on a fluorescence plate reader.

Duplicate data points are generated from a ⅓ log dilution series of a stock solution of test compound in DMSO. Nine dilutions steps are made from a top concentration of 10 μM, and a ‘no compound’ blank is included. Data is collected and analysed using XLfit software from IDBS. The dose response curve is curve fitted to model number 205 (sigmoidal dose-response model). From the curve generated, the concentration giving 50% inhibition is determined and reported.

### LPS-Stimulation of THP-1 Cells

THP-1 cells were plated in 100 μl at a density of 4×10⁴ cells/well in V-bottomed 96 well tissue culture treated plates and incubated at 37° C. in 5% CO₂ for 16 hours. 2 hours after the addition of the inhibitor in 100 μl of tissue culture media, the cells were stimulated with LPS (E coli strain 005:B5, Sigma) at a final concentration of 1 μg/ml and incubated at 37° C. in 5% CO₂ for 6 hours. TNF-α levels were measured from cell-free supernatants by sandwich ELISA (R&D Systems #QTA00B).

### LPS-Stimulation of Human Whole Blood

Whole blood was taken by venous puncture using heparinised vacutainers (Becton Dickinson) and diluted in an equal volume of RPMI1640 tissue culture media (Sigma). 100 μl was plated in V-bottomed 96 well tissue culture treated plates. 2 hours after the addition of the inhibitor in 100 μl of RPMI1640 media, the blood was stimulated with LPS (E coli strain 005:B5, Sigma) at a final concentration of 100 ng/ml and incubated at 37° C. in 5% CO₂ for 6 hours. TNF-α levels were measured from cell-free supernatants by sandwich ELISA (R&D Systems #QTA00B).

IC₅₀ values were allocated to one of three ranges as follows:

### Range A: IC50<500 nM

Range B: 500 nM<IC50<1000 nM

### Range C: IC50>1000 nM

Results Table

| | | | Inhibitor activity |
|---|---|---|---|
| | | Inhibitor activity | versus human |
| Example | Inhibitor activity | versus THP-1 | whole blood TNFα |
| Number | versus IKKβ | TNFα release | release |
| 1 | A | A | A |
| 2 | A | A | C |
| 3 | A | C | C |
| 4 | C | A | C |
| 5 | A | C | B |
| 6 | B | B | A |
| 7 | C | A | C |
| 8 | A | NR | NR |
| 9 | A | NR | NR |
| 10 | A | NR | NR |
| 11 | A | NR | NR |

“NR” indicates “Not Relevant”.
Examples 8-11 are the resultant carboxylic acid analogues of the amino acid esters that are cleaved inside cells. When these carboxylic acids are contacted with the cells, they do not penetrate into the cells and hence do not inhibit TNF-α in this assay.

### Broken Cell Carboxylesterase Assay

Any given compound of the present invention wherein R₁ is an ester group may be tested to determine whether it meets the requirement that it be hydrolysed by intracellular esterases, by testing in the following assay.

### Preparation of Cell Extract

U937 or HCT 116 tumour cells (˜10⁹) were washed in 4 volumes of Dulbeccos PBS (˜1 litre) and pelleted at 525 g for 10 min at 4° C. This was repeated twice and the final cell pellet was re-suspended in 35 ml of cold homogenising buffer (Trizma 10 mM, NaCl 130 mM, CaCl₂ 0.5 mM pH 7.0 at 25° C.). Homogenates were prepared by nitrogen cavitation (700 psi for 50 min at 4° C.). The homogenate was kept on ice and supplemented with a cocktail of inhibitors at final concentrations of:
Leupeptin 1 μM
Aprotinin 0.1 μM
E64 8 μM
Pepstatin 1.5 μM
Bestatin 162 μM
Chymostatin 33 μM

After clarification of the cell homogenate by centrifugation at 525 g for 10 min, the resulting supernatant was used as a source of esterase activity and was stored at −80° C. until required.

### Measurement of Ester Cleavage

Hydrolysis of esters to the corresponding carboxylic acids can be measured using the cell extract, prepared as above. To this effect cell extract (˜30 μg/total assay volume of 0.5 ml) was incubated at 37° C. in a Tris-HCl 25 mM, 125 mM NaCl buffer, pH 7.5 at 25° C. At zero time the ester (substrate) was then added at a final concentration of 2.5 μM and the samples were incubated at 37° C. for the appropriate time (usually 0 or 80 mins). Reactions were stopped by the addition of 3× volumes of acetonitrile. For zero time samples the acetonitrile was added prior to the ester compound. After centrifugation at 12000 g for 5 mins, samples were analysed for the ester and its corresponding carboxylic acid at room temperature by LCMS (Sciex API 3000, HP1100 binary pump, CTC PAL). Chromatography was based on a MeCN (75×2.1 mm) column and a mobile phase of 5-95% acetonitrile in water/0.1% formic acid.

### Human Whole Blood Assay

Human heparinised blood (17-IU/ml) was diluted with an equal volume of RPMI-1640 and then sub-aliquoted into 96 well microtitre wells (100 □l/well). Inhibitors of IKKβ that had been serially diluted in RPMI-1640 were added to the wells (100 □l/well) to give a range of final concentrations (5-10000 nM). After incubation for 2 hours at 37° C., TNF□ production was stimulated for 6 hours at 37° C. by the addition of 10 □l of LPS (E Coli 055:B5) to give a final concentration of 100 ng/ml. Plates were then centrifuged for 3 minutes at 800 g and TNF□ present in the supernatant then measured, using a QuantiGlo Chemiluminescent ELISA (R&D Systems).

TABLE 2

| | Enzyme | IC50 in | |
|---|---|---|---|
| | IC50 | Human | Ratio: |
| | (nM) vs | whole blood | Human whole |
| Compound | IKKβ | (nM) | blood/enzyme |
| 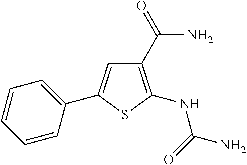 | 5 | 489 | 100 |
| Compound I (parent IKK inhibitor) | | | |
| 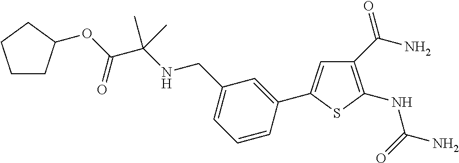 | 132 | 140 | 1 |
| Example 1 | | | |

The human whole blood assay measures the ability of the compounds to inhibit the LPS stimulated production of TNF alpha in human blood cells mediated by IKKβ in a physiologically relevant setting. Table 2 therefore illustrates that conjugation of the parent IKK inhibitor compound to the α,α-disubstituted glycine ester motif which is hydrolysable by an intracellular carboxylesterase (Example 1) leads to a significant decrease in the ratio between potency in cells and the enzyme compared to the parent compound (Compound 1: WO 2004063186) indicating that addition of the esterase motif leads to compounds that show an enhanced level of potency cells.

1. A compound of formula (IA) or (IB), or a salt thereof: 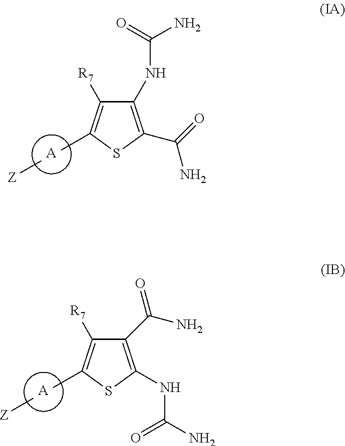
wherein
R₇ is hydrogen or optionally substituted (C₁-C₆)alkyl;
A is optionally substituted aryl or heteroaryl ring or ring system of 5-13 ring atoms;
Z is a radical of formula R₁C(R₂)(R₃)NH—Y-L¹-X¹—(CH₂)_{z}— wherein:
z is 0 or 1;
Y is a bond, —C(═O)—, —S(═O)₂—, —C(═O)NR₇—, —C(═S)—NR₇, —C(═NH)NR₇ or —S(═O)₂NR₇— wherein R₇ is hydrogen or optionally substituted C₁-C₆ alkyl;
L¹ is a divalent radical of formula -(Alk¹)ₘ(Q)ₙ(Alk²)ₚ— wherein
m, n and p are independently 0 or 1,
Q is (i) an optionally substituted divalent mono- or bicyclic carbocyclic or heterocyclic radical having 5-13 ring members, or (ii), in the case where both m and p are 0, a divalent radical of formula —X²-Q¹- or -Q¹-X²— wherein X² is —O—, S— or NR^{A}— wherein R^{A} is hydrogen or optionally substituted C₁-C₃ alkyl, and Q¹ is an optionally substituted divalent mono- or bicyclic carbocyclic or heterocyclic radical having 5-13 ring members,
Alk¹ and Alk² independently represent optionally substituted divalent C₃-C₇ cycloalkyl radicals, or optionally substituted straight or branched, C₁-C₆ alkylene, C₂-C₆ alkenylene, or C₂-C₆ alkynylene radicals which may optionally contain or terminate in an ether (—O—), thioether (—S—) or amino (—NR^{A}—) link wherein R^{A} is hydrogen or optionally substituted C₁-C₃ alkyl; and
X¹ represents a bond; —C(═O); or —S(═O)₂—; —NR₄C(═O)—, —C(═O)NR₄—, —NR₄C(═O)NR₅—, —NR₄S(═O)₂—, or —S(═O)₂NR₄— wherein R₄ and R₅ are independently hydrogen or optionally substituted C₁-C₆ alkyl.
R₁ is a carboxylic acid group (—COOH), or an ester group which is hydrolysable by one or more intracellular esterase enzymes to a carboxylic acid group; and
R₂ and R₃ independently represent the side chain of a natural or non-natural alpha amino acid but neither of R₂ and R₃ is hydrogen, or R₂ and R₃ taken together with the carbon atom to which they are attached form a C₃-C₇ cycloalkyl ring

2. A compound as claimed in claim 1 wherein R₇ is hydrogen.

3. A compound as claimed in claim 1 wherein A is optionally substituted 1,4-phenylene or 1,3-phenylene.

4. A compound as claimed in claim 1 wherein optional substituents in A are selected from, fluoro, chloro, methyl, and trifluoromethyl.

5. A compound as claimed in claim 1 wherein R₁ is an ester group of formula —(C═O)OR₁₄ wherein R₁₄ is R₈R₉R₁₀C— wherein
(i) R₈ is hydrogen, fluorine or optionally substituted (C₁-C₃)alkyl-(Z¹)ₐ-[(C₁-C₃)alkyl]_{b}- or (C₂-C₃)alkenyl-(Z¹)ₐ—[(C₁-C₃)alkyl]_{b}- wherein a and b are independently 0 or 1 and Z¹ is —O—, —S—, or —NR₁₁— wherein R₁₁ is hydrogen or (C₁-C₃)alkyl; and R₉ and R₁₀ are independently hydrogen or (C₁-C₃)alkyl-;
(ii) R₈ is hydrogen or optionally substituted R₁₂R₁₃N—(C₁-C₃)alkyl- wherein R₁₂ is hydrogen or (C₁-C₃)alkyl and R₁₃ is hydrogen or (C₁-C₃)alkyl; or R₁₂ and R₁₃ together with the nitrogen to which they are attached form an optionally substituted monocyclic heterocyclic ring of 5- or 6-ring atoms or bicyclic heterocyclic ring system of 8 to 10 ring atoms, and R₉ and R₁₀ are independently hydrogen or (C₁-C₃)alkyl-; or
(iii) R₈ and R₉ taken together with the carbon to which they are attached form an optionally substituted monocyclic carbocyclic ring of from 3 to 7 ring atoms or bicyclic carbocyclic ring system of 8 to 10 ring atoms, and R₁₀ is hydrogen.

6. A compound as claimed in claim 1 wherein R₁ is a methyl, ethyl, n- or iso-propyl, n-, sec- or tert-butyl, cyclohexyl, allyl, phenyl, benzyl, 2-, 3- or 4-pyridylmethyl, N-methylpiperidin-4-yl, tetrahydrofuran-3-yl, methoxyethyl, indanyl, norbornyl, dimethylaminoethyl, or morpholinoethyl ester group.

7. A compound as claimed in claim 1 wherein R₂ and R₃ are independently phenyl, or heteroaryl or a group of formula —CRₐR_{b}R_{c} in which:
each of Rₐ, R_{b} and R_{c} is independently hydrogen, (C₁-C₆)alkyl, (C₂-C₆)alkenyl, (C₂-C₆)alkynyl, phenyl(C₁-C₆)alkyl, (C₃-C₈)cycloalkyl; or
R_{c} is hydrogen and Rₐ and R_{b} are independently phenyl or heteroaryl such as pyridyl; or
R_{c} is hydrogen, (C₁-C₆)alkyl, (C₂-C₆)alkenyl, (C₂-C₆)alkynyl, phenyl(C₁-C₆)alkyl, or (C₃-C₈)cycloalkyl, and Rₐ and R_{b} together with the carbon atom to which they are attached form a 3 to 8 membered cycloalkyl or a 5- to 6-membered heterocyclic ring; or
Rₐ, R_{b} and R_{c} together with the carbon atom to which they are attached form a tricyclic ring (for example adamantyl); or
Rₐ and R_{b} are each independently (C₁-C₆)alkyl, (C₂-C₆)alkenyl, (C₂-C₆)alkynyl, phenyl(C₁-C₆)alkyl, or a group as defined for R_{c} below other than hydrogen, or Rₐ and R_{b} together with the carbon atom to which they are attached form a cycloalkyl or heterocyclic ring, and R_{c} is hydrogen, —OH, —SH, halogen, —CN, —CO₂H, (C₁-C₄)perfluoroalkyl, —CH₂OH, —O(C₁-C₆)alkyl, —O(C₂-C₆)alkenyl, —S(C₁-C₆)alkyl, —SO(C₁-C₆)alkyl, —SO₂(C₁-C₆) alkyl, —S(C₂-C₆)alkenyl, —SO(C₂-C₆)alkenyl, —SO₂(C₂-C₆)alkenyl or a group -Q-W wherein Q represents a bond or —O—, —S—, —SO— or —SO₂— and W represents a phenyl, phenylalkyl, (C₃-C₈)cycloalkyl, (C₃-C₈)cycloalkylalkyl, (C₄-C₈)cycloalkenyl, (C₄-C₈)cycloalkenylalkyl, heteroaryl or heteroarylalkyl group, which group W may optionally be substituted by one or more substituents independently selected from, hydroxyl, halogen, —CN, —CONH₂, —CONH(C₁-C₆)alkyl, —CONH(C₁-C₆alkyl)₂, —CHO, —CH₂OH, (C₁-C₄)perfluoroalkyl, —O(C₁-C₆)alkyl, —S(C₁-C₆)alkyl, —SO(C₁-C₆)alkyl, —SO₂(C₁-C₆)alkyl, —NO₂, —NH₂, —NH(C₁-C₆)alkyl, —N((C₁-C₆)alkyl)₂, —NHCO(C₁-C₆)alkyl, (C₁-C₆)alkyl, (C₂-C₆)alkenyl, (C₂-C₆)alkynyl, (C₃-C₈)cycloalkyl, (C₄-C₈)cycloalkenyl, phenyl or benzyl.

8. A compound as claimed in claim 1 wherein R₂ and R₃ are independently H-Alk⁴-, phenyl, monocyclic heterocyclyl, C₃-C₇ cycloalkyl, phenyl(Alk⁴)-, heterocyclyl(Alk⁴)-, or C₃-C₇ cycloalkyl(Alk⁴)-, wherein the heterocyclyl part is monocyclic heterocyclyl having 3-7 ring atoms, and wherein -Alk⁴- is a straight or branched, divalent (C₁-C₆)alkylene, (C₂-C₆)alkenylene, or (C₂-C₆)alkynylene radical which may optionally be interrupted by, or terminate in, an ether (—O—), thioether (—S—) or amino (—NR^{A}—) link wherein R^{A} is hydrogen or optionally substituted (C₁-C₃)alkyl, and wherein the Alk⁴-, or cyclic part is optionally substituted.

9. A compound as claimed in claim 1 wherein R₂ and R₃ are independently methyl, ethyl, or n- or iso-propyl, or n, sec or tert-butyl.

10. A compound as claimed in claim 1 wherein at least one of R₂ and R₃ is methyl.

11. A compound as claimed in claim 1 wherein R₂ and R₃ taken together with the carbon atom to which they are attached form a C₃-C₇ cycloalkyl ring, such as cyclopropyl, cyclobutyl, cyclopentyl or cyclohexyl ring.

12. A compound as claimed in claim 1 wherein the radical R₁C(R₂)(R₃)NH—Y-L¹X¹—(CH₂)_{z}— is selected from R₁C(R₂)(R₃)NH—C(═O)—, R₁C(R₂)(R₃)NH—(CH₂)ₐ—, R₁C(R₂)(R₃)NH—(CH₂)ₐO—, and R₁C(R₂)(R₃)NH—CH₂CH═CHCH₂—, wherein a is 1, 2, 3, 4 or 5.

13. A compound as claimed in claim 1 which is
Cyclopentyl N-{3-[4-carbamoyl-5-(carbamoylamino)-2-thienyl]benzyl}-2-methylalaninate or
Cyclopentyl N-(2-{3-[4-carbamoyl-5-(carbamoylamino)-2-thienyl]phenyl}ethyl)-2-methylalaninate
or a salt, N-oxide, hydrate or solvate thereof.

14. A pharmaceutical composition comprising a compound as claimed in claim 1 together with one or more pharmaceutically acceptable carriers and/or excipients.

15. A composition comprising a compound as claimed in claim 1 in an amount for inhibiting the activity of an IKK enzyme.

16. The composition as claimed in claim 15 for the inhibition of IKK-β activity, ex vivo or in vivo.

17. A composition comprising a compound as claimed in claim 1 for treatment of neoplastic/proliferative, immune or inflammatory disease.

18. A method of inhibiting the activity of an IKK enzyme comprising contacting the enzyme with an amount of a compound as claimed in claim 1 effective for such inhibition.

19. A method as claimed in claim 18 for the inhibition of IKK-β activity, ex vivo or in vivo.

20. A method for the treatment of neoplastic/proliferative, immune or inflammatory disease, which comprises administering to a subject suffering such disease an effective amount of a compound as claimed in claim 1.

21. The method as claimed in claim 20 for the treatment of cancer cell proliferation.

22. The method as claimed in claim 20 for the treatment of hepatocellular cancer or melanoma.

23. The method as claimed in claim 20 for the treatment of bowel cancer, ovarian cancer, head and neck and cervical squamous cancers, gastric or lung cancers, anaplastic oligodendrogliomas, glioblastoma multiforme or medulloblastomas.

24. The method as claimed in claim 20 for the treatment of rheumatoid arthritis, psoriasis, inflammatory bowel disease, Crohn's disease, ulcerative colitis, chronic obstructive pulmonary disease, asthma, multiple sclerosis, diabetes, atopic dermatitis, graft versus host disease, systemic lupus erythematosus, Type II diabetes mellitus or Alzheimers disease.
